SEQUENCE LISTING (5 sequences)

SEQ ID NO: 1 - DNA, 600 bp, Homo sapiens
ttttccatttccttcttaaggtcactgaaatgtgctccttggagccagcccgcaaatcacgcatttagaaaaacataactatacactcctaaccctaagtattagaagtgaaagtaatggaatctcgatgtaaacacaatatcacttttttgatgagctattttgagtataataaatttgaactgtgccaatgctgggagaaaaaatttaaaagaagaacggagcgaacagtagcttcctgctccgctgactagaaacagtaggacgacactctcccgactggaggagagcgcttgcgctcgcactcagttggcgcccgccctcctgctttttctctagccgccctttcctctttctttcgcgctctagccacccgggaaggcctgcccagcgtagctgggctctgattggctgctttgaaagtctacgggctacccgattggtgaatccggggccctttagcgcggtgagtttgaaactgctcgcacttggcttcaaagctggctcttggaaattgagcggagagcgacgcggttgttgtagctgccgctgcggccgccgcggaataataagccgggtacagtggctggggtcagggtc

SEQ ID NO: 2 - DNA, 3152 bp, Homo sapiens
atatctaagatttgtcccaagagaaactgcagatcccagagtctgacatatataaagcacaagtaggcaatagctagcatcagttaaatggcctctattaacaagtgttattaagaaattaggtttgttatgggcaaatggattaacactataaataatttattttttgtagtataaattattttgaagcaaagagtatcatccattattcctttcacttcaaagatcaccttgagattaaacattttctgcaaaattttgataaaaacacacaaattattatttccaaaggacttttcctgctcacccctccttcctcacaatcaatacaactaaaactaaaatgagattggatttagtgagctgtccagtgactcaatatccctagggcaaagaaatagtacaaactgcaaagagctgtttacagaagctactttaatacatgggtaagagtaaacagccatggtacaactatttacttgtaggaggaaaagtgtcactttctggtatcagatgctaatccagactggttttatagaaacaaaattataaaactggaagagacgttatgtaatcacaatatttaagactgcatcttcaaatagcactaagtgatatataagtgagttaaataaaactgaaatctattctcccaaccaagaacacatttatgggatcagattaaaataagcatttaagatctaattataattagattgatgtgactggcagtgaacctgaaagagcactggataaggatctttttgtatgtcaccaagtcaagtgttagctgtgagatctgagataagtcacatcctttctaagccttgttacattaaacctaactcactatagtcaaaatgactacataagacatcttaaaatgcaaatcattaatatcatctaataattataattaccgtaattccacacagattttccttaaaacttaccaaaactaccatccattggataatcaagagggaccaatggttttctgggtccaggtaaactaatggctgaattaaaagccagggcatcttcacgctctattttttgagattcagctggcttcttctgagcttctttttctgcttcatccacatgaatggtgaacgcaggctgtttactgtttgctttccaaggaggaacggtgacatgctcatcatttacaggaagatccttaaggggtgcaacctaaaaaaaaattaataactggctttgagcctactagtcttgcattctttctcttatgggtgttggcctttgctttttctcccaaatactctatcaggaattaaactttctgggacaaagaagagtttcagtattcaaaagagaagggatgttcccatttgtcacaggcattcccacaaaggctatcaatagataacactaggtggaattatgcaaaacttatcagggccacaagaagggtcgggtgacaggccaatacgtgacaagtttaggaaagacagcaaaaaaattgttggttcaccactgtcgcccgagggtggagcaacagagcaaatccggctaaaatggcggaatttgccaaaagcaaagtattttgtacacgcttttctaaagtagaggtcatctcttaagcgtgaggaggataggggtcagagtcctacagcagtgcctggggtttaaaagtggagagaaaggcgcggagctgagcgaagactacacgttttagagcttggtttgaccccattatgtacaggcagatgccccctatagagagcagaccggcagcatacacacagtgttggcggggtcctgatgctaatatgggagaactcctactgtggcaaaccacacaccagcaccaactgcactccagaccctggcccccttccaaaagcccaggacatgccttctcgccactcttctctgcctgctaagccaatgccaaaccctgctcgacccaccctcctgcagatatcccgcatccctttacccgtctcgtcttcggcctctgctgctgcgctagaccccgcgggttcccggacttcagtaccgccagcgcggcccgggtccgcggttgttggacgggcgctgccttttccgggttgatattctcctggtcctcttggagcgccgtctgctgcaatgctagcagcgccgagcccgcctcgcgggtcgcaggccccggcgcagagttgcccaacatcactgctcccgggagtggacggcgggatcagcctgcggcgccaagcagcgtgcactctgcccagccgaccactcgcaccgacccggccaaagaatagtcgtagccgccggtcgcagcccaggccagcctaccagcccgcccgctcgctcacccagctcgagaccacgcagggccgaggaggttgcgaaaggcgcaggcaggcactgcccagcgtggcgagccaaagacgcccagagatgcagcgagcagcccgccggagcggcggctgttcttgcagttcaagtatcccgcgactattgaaatggaccaatgaaagcgctcgcggccttgacgtcattcaaggcgacagggtcgcaggcgagtgaagggtaaaccaaaggaaactgagcaggggcggggcaggagggagaaacaaactggctggggcgggagaaaacgcctgcgcggggcggggagaggtaggatttagggcccgacgcttccgattattttaagctgagccacctagtgagcgaggctgtccgaaggctgactctaagccttttagggaattaaacggaattaaactttggcgactatcatttgggttgcccagcctttagctctgggacgtctagttagcttaagtgactttttccaaaggtctaaaatctggggcacgtttttcagtatctccgattctccgttttcttatgtgttaatagggactaataactacagtcatatggttgttaggattaaataatgtgcataaattatttaacacggagctcacatagtaatacagtactcaagtgtcaagactgtaatgtcatcttaacatttaggcgtttattcatagtaaccagaaattgtgttttatgctttttatttttcctaatgaagatgcaattaatttaagatggcaccttgaactactgttgatatatataataaatagcagtgcattacaaaatattcaaatacagtcatt

SEQ ID NO: 3 - DNA, 3001 bp, Homo sapiens
tgaaatgctctatactttctgctaagttttgctgcgaacctaaaactgctctaaaaaataaagcatataaaaggaaaaatttggccgggcgtggtggctcacgcctgtaatcgcagcactttgggaggccgaggcgggcggatcaccaagtcaggagatcgagaccatcctggctaacatggtgaaaccccgtctctaccaaaaatacaaaaaattagccggccgttgtggcgggcgcctgccgtcccagctactcgggaggctgaggcaggagaatggagtgaaccctggaggtggaggttgcagtgagtcgagatcgcaccactgcactccagcctgggcgacagatcgagactccgtctcaaaaaaaagtaaaaaaaaaaatttttaaactggaacacagaggataagtaatcctgcttcgccccttgcctctcgcccctgcatggggcgaggaaggattgatcaaacccagaaagactgagtgagaatggatgttgaacacagaactgaaggatgaatgggagttacctgaatagaacccaggggcttgaatgcaagaagaggcgggcattccaggcagaggagagcaagggtaagggccccacgggaggcattcgagtaggagggtgaactatgatgacagaagactcaataacgatccaaagaaaccaaatgattgggcgccttctttcggatccgtgacttccagcgccaggagtctctattggctcttataccgttgctctatgggatagcaatgtttttgtccttcagcctcccctccaattgctgagctgctggtgtgttttgaggagtagaaggcaaaaagaaccctcttgtttttctttggatctagagagaatctgagcaaatgacaaagcaaatggggtaaaatgtctttttgtttagtttttcctgattttcccatgagaggcaaatacatgttaaggatagttgaatctgagtaaagggcatagaaatattccttacaagatttttgttggcaactggtctaagtatgaaattatttccaaatagaaagctaaaacaaacaaaacaattggccttgggaaactggacaatcttgaagtaatcaagtaatattgttaagggacaatcagtgttgtgaaaacaacacggatacaccccctccctccccctcaaaaaaaaaaaccctaaattcagttcccccgttgctaatgtgtgaccctggcaaagtcatctaagtcgctgagcttcagttcctccaacccagagagttgttgcaacgatcaaatgaaagaatgtctattaaagcctttcatgaactatattattgctgctaccgtagaaatggaaagtgtgcaacactagatccaaaactacttttgacacttctgagactgtggccgcgcctctgtcaccttccaaaggccactaggcctttcctgagctggcattggcaacgcacactcttgcccggctaacctttccaggtgggcggcgcactggcttcactgctctccaggtggccgctgcagctgcccgagagcgcaggcgcagaggcagaccacgtgagagcctggccaggccttccggcctagcctcactgtggccccgcccctctcgaacgccttcgcgcgatcgccctggaaacgcattctctgcgaccggcagccgccaatgggaagggagtgagtgccacgaacaggccaataaggagggagcagtgcggggtttaaatctgaggctaggctggctcttctcggcgtgctgcggcggaacggctgttggtttctgctgggtgtaggtccttggctggtcgggcctccggtgttctgcttctccccgctgagctgctgcctggtgaagaggaagccatggcgctccgagtcaccagggtgagccgcttcggactgcgaactaacgcggccttcttagctgctgcctgctctccctgcctcgcctgcgggagcctcccgagcgggagagggccgcaggagcgatttggggaggaaggtgggaggggactcaccaagagagcgccgaggtgggcccaggcctggtgagagagtgtgggggacgatggatggagggaggaaggtgagaaagagaactggacggatattggataaatgttttggggaggtggagagtcgactgggaaccttttgaaaaagtgatagagggtccctgagtgggcccgccagcaactctgtaacccccttcccagagagaaggtgtctgcaattggaggctttttcggtttcctttcaaatgtaaattctcggtattttagggctggccaggactaatcagggaatccctcaattggtaaatgtagaggtcggcggaaactgacttgtcacggccgcagagtagactctgggacccatgttttccctcggaacccatttttagtcggctttctttctgggaacttctccttgtgccccaccttaattaacccttgacttactcgagccttcgtggatcagctcttaaagtggtcttgcttctttcagaactcgaaaattaatgctgaaaataaggcgaagatcaacatggcaggcgcaaagcgcgttcctacggcccctgctgcaacctccaagcccggactgaggccaagaacagctcttggggacattggtaacaaagtcagtgaacaactgcaggccaaaatgcctatgaagaaggtaactctcttcctgacctaacttctgtaagagcccgccttccaactgtggcctttgatgcagaaacatttcattctctctgtttcatctacaggaagcaaaaccttcagctactggaaaagtcattgataaaaaactaccaaaacctcttgaaaaggtacctatgctggtgccagtgccagtgtctgagccagtgccagagccagaacctgagccagaacctgagcctgttaaagaagaaaaactttcgcctgagcctattttggtaaacttattcttaccattgtagagtctgttgattatttcttgtcccttatttcact

SEQ ID NO: 4 - DNA, 23 bp, Artificial Sequence
  Synthetic Oligonucleotide
gtacatcttctgtgtgatcttgg

SEQ ID NO: 5 - DNA, 20 bp, Artificial Sequence
  Synthetic Oligonucleotide
gtacatcttctgtgtgatct

(19) United States
(12) Patent Application Publication
(10) Pub. No.: US 2017/0058272 A1
(43) Pub. Date: Mar. 2, 2017

(54) DIRECTED NUCLEIC ACID REPAIR

(71) Applicant: Caribou Biosciences, Inc., Berkeley, CA (US)

(72) Inventors: Matthew M. Carter, Berkeley, CA (US); Megan Van Overbeek, Oakland, CA (US); Andrew Paul May, San Francisco, CA (US)

(21) Appl. No.: 15/253,725

(22) Filed: Aug. 31, 2016

Related U.S. Application Data
(60) Provisional application No. 62/212,517, filed on Aug. 31, 2015.

(51) Int. Cl. C12N 9/22; C12N 7/00; C12N 15/86

(52) U.S. Cl. CPC C12N 9/22; C12N 15/86; C12Y 301/00; C12N 7/00; C12N 2740/15043

(57) ABSTRACT

The present disclosure provides compositions and methods for enhancing directed nucleic acid repair, which are useful in the area of genome engineering.

### CROSS-REFERENCE TO RELATED APPLICATIONS

This application claims the benefit of U.S. Provisional Patent Application Ser. No. 62/212,517, filed 31 Aug. 2015, now pending, which application is herein incorporated by reference in its entirety.

### STATEMENT REGARDING FEDERALLY SPONSORED RESEARCH OR DEVELOPMENT

Not applicable.

### SEQUENCE LISTING

The present application contains a Sequence Listing that has been submitted electronically in ASCII format and is hereby incorporated by reference in its entirety. The ASCII copy, created on 31 Aug. 2016, is named CBI018-10_ST25.txt and is 10 KB in size.

### TECHNICAL FIELD

The present disclosure relates generally to the area of genome engineering. In particular, the disclosure relates to compositions and methods for directed nucleic acid repair.

### BACKGROUND

Genome engineering includes altering the genome by deleting, inserting, mutating, or substituting specific nucleic acid sequences. The alteration can be gene or location specific. Genome engineering can use nucleases to cut DNA, thereby generating a site for alteration. In certain cases, the cleavage can introduce a double-strand break (DSB) in the target DNA. DSBs can be repaired, e.g., by non-homologous end joining (NHEJ), microhomology-mediated end joining (MMEJ), or homology-directed repair (HDR). HDR relies on the presence of a template for repair. In some examples of genome engineering, a donor polynucleotide or portion thereof can be inserted into the break.

Clustered regularly interspaced short palindromic repeats (CRISPR) and CRISPR associated proteins (Cas) constitute the CRISPR-Cas system. This system provides adaptive immunity against foreign DNA in bacteria (Barrangou, R., et al., Science 315:1709-1712 (2007); Makarova, K. S., et al, Nat Rev Microbiol 9:467-477 (2011); Garneau, J. E., et al, Nature 468:67-71 (2010); Sapranauskas, R., et al., Nucleic Acids Res 39:9275-9282 (2011)).

CRISPR-Cas systems have recently been reclassified into two classes, comprising five types and sixteen subtypes (Makarova, K., et al., Nature Reviews Microbiology 13:1-15 (2015)). This classification is based upon identifying all cas genes in a CRISPR-Cas locus and determining the signature genes in each CRISPR-Cas locus, ultimately determining that the CRISPR-Cas systems can be placed in either Class 1 or Class 2 based upon the genes encoding the effector module, i.e., the proteins involved in the interference stage. Recently a sixth CRISPR-Cas system has been identified (Abudayyeh O., et al., Science 353(6299):aaf5573 (2016)).

Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III, and Type IV systems. Class 2 systems comprise Type II and Type V systems.

Type II systems have cas1, cas2, and cas9 genes. The cas9 gene encodes a multidomain protein that combines the functions of the crRNA-effector complex with target DNA cleavage. Type II systems also encode a tracrRNA. Type II systems are further divided into three sub-types, sub-types II-A, II-B, and II-C. Sub-type II-A contains an additional gene, csn2. An example of an organism with a sub-type II-A system is Streptococcus thermophilus. Sub-type II-B lacks csn2, but has cas4. An example of an organism with a sub-type II-B system is Legionella pneumophila. Sub-type II-C is the most common Type II system found in bacteria and has only three proteins, Cas1, Cas2, and Cas9. An example of an organism with a sub-type II-C system is Neisseria lactamica.

Type V systems have a cpf1 gene and cas1 and cas2 genes. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3Cpf1), Butyrivibrio proteoclasticus (BpCpf1), Peregrinibacteria bacterium GW2011_WA_33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237(MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity, and it is responsible for pre-crRNA processing (Fonfara, I., et al., Nature 532(7600):517-521 (2016)).

In Class 2 systems, the crRNA is associated with a single protein and achieves interference by combining nuclease activity with RNA-binding domains and base-pair formation between the crRNA and a target nucleic acid sequence.

In Type II systems, target binding involves Cas9 and the crRNA, as does the target nucleic acid sequence cleavage. In Type II systems, the RuvC-like nuclease (RNase H fold) domain and the HNH (McrA-like) nuclease domain of Cas9 each cleave one of the strands of the double-stranded target nucleic acid sequence. The Cas9 cleavage activity of Type II systems also requires hybridization of crRNA to tracrRNA to form a duplex that facilitates the crRNA and target binding by the Cas9.

In Type V systems, target binding involves Cpf1 and the crRNA, as does the target nucleic acid sequence cleavage. In Type V systems, the RuvC-like nuclease domain of Cpf1 cleaves one strand of the double-stranded target nucleic acid sequence, and a putative nuclease domain cleaves the other strand of the double-stranded target nucleic acid sequence in a staggered configuration, producing 5′ overhangs, which is in contrast to the blunt ends generated by Cas9 cleavage. These 5′ overhangs may facilitate insertion of DNA.

The Cpf1 cleavage activity of Type V systems also does not require hybridization of crRNA to tracrRNA to form a duplex, rather the crRNA of Type V systems uses a single crRNA that has a stem-loop structure forming an internal duplex. Cpf1 binds the crRNA in a sequence and structure specific manner that recognizes the stem loop and sequences adjacent to the stem loop, most notably the nucleotide 5′ of the spacer sequences that hybridizes to the target nucleic acid sequence. This stem-loop structure is typically in the range of 15 to 19 nucleotides in length. Substitutions that disrupt this stem-loop duplex abolish cleavage activity, whereas other substitutions that do not disrupt the stem-loop duplex do not abolish cleavage activity. In Type V systems, the crRNA forms a stem-loop structure at the 5′ end, and the sequence at the 3′ end is complementary to a sequence in a target nucleic acid sequence.

Other proteins associated with Type V crRNA and target binding and cleavage include Class 2 candidate 1 (C2c1) and Class 2 candidate 3 (C2c3). C2c1 and C2c3 proteins are similar in length to Cas9 and Cpf1 proteins, ranging from approximately 1,100 amino acids to approximately 1,500 amino acids. C2c1 and C2c3 proteins also contain RuvC-like nuclease domains and have an architecture similar to Cpf1. C2c1 proteins are similar to Cas9 proteins in requiring a crRNA and a tracrRNA for target binding and cleavage but have an optimal cleavage temperature of 50° C. C2c1 proteins target an AT-rich protospacer adjacent motif (PAM), which similar to Cpf1, is 5′ of the target nucleic acid sequence (see, e.g., Shmakov, S., et al., Molecular Cell 60(3):385-397 (2015)).

Class 2 candidate 2 (C2c2) does not share sequence similarity to other CRISPR effector proteins and was recently identified as a Type VI system (Abudayyeh O., et al., Science 353(6299):aaf5573 (2016)). C2c2 proteins have two HEPN domains and demonstrate single-stranded RNA-cleavage activity. C2c2 proteins are similar to Cpf1 proteins in requiring a crRNA for target binding and cleavage, while not requiring tracrRNA. Also similar to Cpf1, the crRNA for C2c2 proteins forms a stable hairpin, or stem-loop structure, that aids in association with the C2c2 protein.

Regarding Class 2 Type II CRISPR-Cas systems, a large number of Cas9 orthologs are known in the art as well as their associated polynucleotide components (tracrRNA and crRNA) (see, e.g., Fonfara, I., et al., Nucleic Acids Research 42(4):2577-2590 (2014), including all Supplemental Data; Chylinski K., eta′, Nucleic Acids Research 42(10):6091-6105 (2014), including all Supplemental Data). In addition, Cas9-like synthetic proteins are known in the art (see U.S. Published Patent Application No. 2014-0315985, published 23 Oct. 2014).

Cas9 is an exemplary Type II CRISPR Cas protein. Cas9 is an endonuclease that can be programmed by the tracrRNA/crRNA to cleave, site-specifically, a target DNA sequence using two distinct endonuclease domains (HNH and RuvC/RNase H-like domains) (see U.S. Published Patent Application No. 2014-0068797, published 6 Mar. 2014; see also Jinek M., et al., Science 337:816-821 (2012)).

Typically, each wild-type CRISPR-Cas9 system includes a tracrRNA and a crRNA. The crRNA has a region of complementarity to a potential DNA target sequence and a second region that forms base-pair hydrogen bonds with the tracrRNA to form a secondary structure, typically to form at least a stem structure. The region of complementarity to the DNA target is the spacer. The tracrRNA and a crRNA interact through a number of base-pair hydrogen bonds to form secondary RNA structures. Complex formation between tracrRNA/crRNA and Cas9 protein results in conformational change of the Cas9 protein that facilitates binding to DNA, endonuclease activities of the Cas9 protein, and crRNA-guided site-specific DNA cleavage by the endonuclease Cas9. For a Cas9 protein/tracrRNA/crRNA complex to cleave a double-stranded DNA target sequence, the DNA target sequence is adjacent to a cognate PAM. By engineering a crRNA to have an appropriate spacer sequence, the complex can be targeted to cleave at a locus of interest, e.g., a locus at which some type of sequence modification is desired.

Ran, F. A., et al., Nature 520(7546):186-191 (2015), including all extended data, present the crRNA/tracrRNA sequences and secondary structures of eight Type II CRISPR-Cas systems (see Extended Data Figure 1 of Ran, F. A., et al.). Predicted tracrRNA structures were based on the Constraint Generation RNA folding model (Zuker, M., Nucleic Acids Res. 31:3406-3415 (2003)). Furthermore, Fonfara, et al., Nucleic Acids Research 42(4):2577-2590 (2014), including all Supplemental Data (in particular Supplemental Figure S11) present the crRNA/tracrRNA sequences and secondary structures of eight Type II CRISPR-Cas systems. RNA duplex secondary structures were predicted using RNAcofold of the Vienna RNA package (Bernhart, S. H., et al., Algorithms Mol. Biol. 1(1):3 (2006); Hofacker, I. L., et al., J. Mol. Biol. 319:1059-1066 (2002)) and RNAhybrid (bibiserv.techfak.uni-bielefeld.de/rnahybrid/). The structure predictions were visualized using VARNA (Darty, K., et al., VARNA: Interactive drawing and editing of the RNA secondary structure Bioinformatics 25:1974-1975 (2009)). Fonfara, et al., show that the crRNA/tracrRNA complex for Campylobacter jejuni does not have the bulge region; however, it retains a stem structure located 3′ of the spacer that is followed in the 3′ direction with another stem structure.

Naturally occurring Type V CRISPR-Cas systems, unlike Type II CRISPR Cas systems, do not require a tracrRNA for crRNA maturation and cleavage of a target nucleic acid sequence. In a typical structure of a crRNA from a Type V CRISPR system, the DNA target-binding sequence is downstream of a specific secondary structure (i.e., a stem-loop structure) that interacts with the Cpf1 protein. The bases 5′ of the stem loop adopt a pseudoknot structure further stabilizing the stem-loop structure with non-canonical Watson-Crick base-pairing (e.g., U base-pairs with U) and a triplex interaction involving reverse Hoogsteen base-pairing (e.g., U base-pairs with A base-pairs with U).

To date, two Type V CRISPR Cas systems, from Acidaminococcus and Lachnospiraceae, have demonstrated genome-editing activity in human cells (Zetsche, Bernd, et al., Cell 163:759-771 (2015)).

The spacer of Class 2 CRISPR-Cas systems can hybridize to a target nucleic acid that is located 5′ or 3′ of a PAM, depending upon the Cas protein to be used. A PAM can vary depending upon the Cas polypeptide to be used. For example, when using the Cas9 from S. pyogenes, the PAM can be a sequence in the target nucleic acid that comprises the sequence 5′-NRR-3′, wherein R can be either A or G, wherein N is any nucleotide, and N is immediately 3′ of the target nucleic acid sequence targeted by the targeting region sequence. A Cas protein may be modified such that a PAM may be different compared with a PAM for an unmodified Cas protein. For example, when using Cas9 protein from S. pyogenes, the Cas9 protein may be modified such that the PAM no longer comprises the sequence 5′-NRR-3′, but instead comprises the sequence 5′-NNR-3′, wherein R can be either A or G, wherein N is any nucleotide, and N is immediately 3′ of the target nucleic acid sequence targeted by the targeting region sequence.

Other Cas proteins recognize other PAMs, and one of skill in the art is able to determine the PAM for any particular Cas protein. For example, Cpf1 has a thymine-rich PAM site that targets, for example, a TTTN sequence (Fagerlund, R., et al., Genome Biol. 16:251 (2015)).

The RNA-guided Cas9 endonuclease has been widely used for programmable genome editing in a variety of organisms and model systems (Jinek M., et al., Science 337:816-821 (2012); Jinek M., et al., Elife 2:e00471. doi: 10.7554/eLife.00471 (2013); U.S. Published Patent Application No. 2014-0068797, published 6 Mar. 2014).

There is a need for improved targeted DNA repair, particularly in genome engineering. This need can be addressed by using engineered Class 2 CRISPR-Cas compositions described herein.

### SUMMARY OF THE INVENTION

In one aspect, the present invention relates to engineered Class 2 CRISPR-Cas compositions that confer conditional expression of a Cas protein in response to particular cell states. Additional aspects of the present invention relate to vectors, kits, host cells, and methods comprising these engineered Class 2 CRISPR-Cas compositions.

In a first aspect, the present invention relates to a Class 2 CRISPR-Cas polynucleotide composition comprising a first polynucleotide encoding a Cas protein, wherein the first polynucleotide is operably linked to a first regulatory element that is active in response to a first cell state of a eukaryotic host cell. In some embodiments, the compositions comprise a locus-specific guide polynucleotide encoding a locus-specific guide RNA capable of forming a complex with the Cas protein. In preferred embodiments, the locus-specific guide polynucleotide is operably linked to a regulatory element that is active in response to the first cell state of the eukaryotic host cell. Examples of such regulatory elements include, but are not limited to, regulatory elements associated with proteins preferentially expressed during a particular cell cycle phase, that is, G₀, G₁, S, G₂, or M.

In one embodiment, the first regulatory element is operably linked to a single polynucleotide comprising the first polynucleotide and the locus-specific guide polynucleotide, wherein a transcript separator sequence is located between the first polynucleotide and the locus-specific guide polynucleotide. Examples of transcripts include self-cleaving ribozymes or sequences recognized by a ribonuclease (e.g., Csy4).

In further embodiments, the first cell state is a transient cell state of the eukaryotic host cell.

In another aspect, the Class 2 CRISPR-Cas polynucleotide compositions of the first aspect of the present invention further comprise a Cas protein-specific guide polynucleotide encoding a Cas protein-specific guide RNA that is capable of targeting the Cas protein to the first polynucleotide. Typically, the Cas protein-specific guide polynucleotide is operably linked to a second regulatory element that is active in response to a second cell state of the eukaryotic host cell. In some embodiments, the first cell state and the second cell state are different. The Cas protein-specific guide polynucleotide can encode multiple copies of the Cas protein-specific guide RNA. Typically, the sequences encoding the copies of the Cas protein-specific guide RNA are separated by a transcript separator sequence. Alternatively, expression of the sequences encoding the Cas protein-specific guide RNAs can be under the control of two or more second regulatory elements.

In yet another aspect of the Class 2 CRISPR-Cas polynucleotide compositions of the first aspect of the present invention, the Class 2 CRISPR-Cas polynucleotide compositions further comprise a repressor polynucleotide encoding a repressor protein that is capable of repressing transcription mediated by the first regulatory element. In some embodiments, the repressor polynucleotide is operably linked to a NHEJ pathway-specific regulatory element that is capable of mediating expression of a protein that drives the NHEJ pathway. For example, the first regulatory element can further comprise a lacO operator sequence, and the repressor polynucleotide can comprise a lac repressor protein coding sequence.

In another aspect of the Class 2 CRISPR-Cas polynucleotide compositions of the first aspect of the present invention, the compositions further comprise a second polynucleotide encoding an inactive Cas (dCas) protein operably linked to a second regulatory element, a locus-specific guide polynucleotide encoding a locus-specific guide RNA capable of forming a complex with the dCas protein, and a locus-specific guide polynucleotide encoding a locus-specific guide RNA capable of forming a complex with the Cas protein. In some embodiments, the first regulatory element and the second regulatory element are both active in response to the first cell state of the eukaryotic host cell. Further embodiments include a locus-specific guide RNA capable of forming a complex with the dCas protein that comprises a NHEJ pathway-specific guide RNA that can target a gene that encodes a protein that drives the NHEJ pathway. In other embodiments, the first cell state comprises a cell cycle phase conducive to HDR (e.g., the cell cycle phase is S or G₂) or a cell cycle phase conducive to NHEJ (e.g., the cell cycle phase is G₁, G₀, or M).

In some embodiments, the Cas protein of the Class 2 CRISPR-Cas polynucleotide compositions of the present invention is a Cas9 protein, and in other embodiments a Cpf1 protein, or a combination thereof.

The Class 2 CRISPR-Cas polynucleotide composition of the present invention can further comprise one or more donor polynucleotides.

In some aspects of the present invention, one or more vectors comprise a Class 2 CRISPR-Cas polynucleotide composition. Examples of vectors useful in the embodiments of the present invention include insect cell vectors for insect cell transformation and gene expression in insect cells, yeast plasmids for cell transformation and gene expression in yeast and other fungi, mammalian vectors for mammalian cell transformation and gene expression in mammalian cells or mammals, viral vectors (including retroviral, lentivirus, adenoviral, adeno-associated, and herpes simplex virus vectors) for cell transformation and gene expression, and plant vectors for cell transformation and gene expression in plants. In a preferred embodiment, a lentiviral vector comprises a Class 2 CRISPR-Cas polynucleotide composition.

The present invention also includes kits comprising the Class 2 CRISPR-Cas polynucleotide compositions described herein. Typically a kit comprise one or more of the following: a buffer, a preservative, and/or instructions for using the Class 2 CRISPR-Cas polynucleotide compositions of the invention.

In further aspects, the present invention includes a host cell comprising the Class 2 CRISPR-Cas polynucleotide compositions described herein.

The present invention also includes a method of directing DNA repair at a locus in a eukaryotic host cell genome. The method typically comprises introducing one or more vectors comprising a Class 2 CRISPR-Cas polynucleotide composition of the present invention and a donor polynucleotide into the eukaryotic host cell. In preferred embodiments, the composition comprises a first polynucleotide encoding a Cas protein, wherein the first polynucleotide is operably linked to a first regulatory element that is active in response to a first cell state of the eukaryotic host cell. This regulatory element is typically active in response to a cell cycle phase S or G₂. At least a portion of the donor polynucleotide is incorporated into the locus to repair the DNA at the locus. In some embodiments of the method, the one or more vectors are introduced into host cell in vivo or ex vivo. In some in vivo embodiments, the host cell is a non-human cell.

These aspects and other embodiments of the present invention using the engineered Class 2 CRISPR-Cas systems of the present invention will readily occur to those of ordinary skill in the art in view of the disclosure herein.

### BRIEF DESCRIPTION OF THE FIGURES

The figures are not proportionally rendered, nor are they to scale. The locations of indicators are approximate.

FIG. 1A, FIG. 1B, FIG. 1C, and FIG. 1D present illustrative examples of Class 2 CRISPR-associated guide RNAs.

FIG. 2, FIG. 3, FIG. 4, and FIG. 5 provide exemplary embodiments of the present invention described with reference to a Class 2 Type II CRISPR-Cas system using a Cas9 protein. These embodiments can also comprise a Type V CRISPR-Cpf1 system using a Cpf1 protein and a Cpf1-specific guide polynucleotide, or combinations of a Cas9 protein/a Cas9-specific guide polynucleotide and a Cpf1 protein/a Cpf1-specific guide polynucleotide.

### INCORPORATION BY REFERENCE

All patents, publications, and patent applications cited in this specification are herein incorporated by reference as if each individual patent, publication, or patent application was specifically and individually indicated to be incorporated by reference in its entirety for all purposes.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can employ conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, Cold Spring Harbor Laboratory Press, ISBN 978-1-936113-81-1 (2014); Culture of Animal Cells: A Manual of Basic Technique and Specialized Applications, 6th Edition, R. I. Freshney, Wiley-Blackwell, ISBN 978-0-470-52812-9 (2010); Transgenic Animal Technology, Third Edition: A Laboratory Handbook, C. A. Pinkert, Elsevier, ISBN 978-0124104907 (2014); The Laboratory Mouse, Second Edition, H. Hedrich, Academic Press, ISBN 978-0123820082 (2012); Manipulating the Mouse Embryo: A Laboratory Manual, R. Behringer, et al., Cold Spring Harbor Laboratory Press, ISBN 978-1936113019 (2013); PCR 2: A Practical Approach, M. J. McPherson, eta′, IRL Press, ISBN 978-0199634248 (1995); Methods in Molecular Biology (Series), J. M. Walker, ISSN 1064-3745, Humana Press; RNA: A Laboratory Manual, D. C. Rio, eta′, Cold Spring Harbor Laboratory Press, ISBN 978-0879698911 (2010); Methods in Enzymology (Series), Academic Press; Molecular Cloning: A Laboratory Manual (Fourth Edition), M. R. Green, et al, Cold Spring Harbor Laboratory Press, ISBN 978-1605500560 (2012); Bioconjugate Techniques, Third Edition, G. T. Hermanson, Academic Press, ISBN 978-0123822390 (2013); Methods in Plant Biochemistry and Molecular Biology, W. V. Dashek, CRC Press, ISBN 978-0849394805 (1997); Plant Cell Culture Protocols (Methods in Molecular Biology), V. M. Loyola-Vargas, et al, Humana Press, ISBN 978-1617798177 (2012); Plant Transformation Technologies, C. N. Stewart, et al, Wiley-Blackwell, ISBN 978-0813821955 (2011); Recombinant Proteins from Plants (Methods in Biotechnology), C. Cunningham, et al, Humana Press, ISBN 978-1617370212 (2010); Plant Genomics: Methods and Protocols (Methods in Molecular Biology), D. J. Somers, et al, Humana Press, ISBN 978-1588299970 (2009); Plant Biotechnology: Methods in Tissue Culture and Gene Transfer, R. Keshavachandran, et al, Orient Blackswan, ISBN 978-8173716164 (2008).

Clustered regularly interspaced short palindromic repeats (CRISPR) and associated Cas proteins constitute CRISPR-Cas systems (Barrangou, R., et al., Science 315:1709-1712 (2007)).

As used herein, “Cas protein” and “CRISPR-Cas protein” refer to CRISPR-associated proteins (Cas) including, but not limited to Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. In a preferred embodiment, a Cas protein is a Class 2 CRISPR-associated protein, for example a Class 2 Type II CRISPR-associated protein, such as Cas9, or a Class 2 Type V CRISPR-associated protein, such as Cpf1. Each wild-type CRISPR-Cas protein is capable of interacting with one or more cognate polynucleotide (most typically RNA) to form a nucleoprotein complex (most typically a ribonucleoprotein complex).

“Cas9 protein,” as used herein, refers to a Cas9 wild-type protein derived from Class 2 Type II CRISPR-Cas9 systems, modifications of Cas9 proteins, variants of Cas9 proteins, Cas9 orthologs, and combinations thereof. Cas9 nucleases are known, for example, Cas9 from Streptococcus pyogenes (UniProtKB—Q99ZW2 (CAS9_STRP1)), Streptococcus thermophilus (UniProtKB—G3ECR1 (CAS9_STRTR)), and Staphylococcus aureaus (sequence: UniProtKB—J7RUA5 (CAS9_STAAU)). “dCas9,” as used herein, refers to variants of Cas9 protein that are nuclease-deactivated Cas9 proteins, also termed “catalytically inactive Cas9 protein,” “enzymatically inactive Cas9,” “catalytically dead Cas9” or “dead Cas9.” Such molecules lack all or a portion of endonuclease activity and can therefore be used to regulate genes in an RNA-guided manner (Jinek M., et al., Science 337:816-821 (2012)). This is accomplished by introducing mutations that inactivate Cas9 nuclease function and is typically accomplished by mutating both of the two catalytic residues (D10A in the RuvC-1 domain, and H840A in the HNH domain, numbered relative to S. pyogenes Cas9). It is understood that mutation of other catalytic residues to reduce activity of either or both of the nuclease domains can also be carried out by one skilled in the art. The resultant dCas9 is unable to cleave double-stranded DNA but retains the ability to complex with a guide nucleic acid and bind a target DNA sequence. The Cas9 double mutant with changes at amino acid positions D10A and H840A completely inactivates both the nuclease and nickase activities. Targeting specificity is determined by complementary base-pairing of guide RNA (typically, an sgRNA) to the genomic locus and the PAM.

“Cpf1 protein,” as used herein, refers to a Cpf1 wild-type protein derived from Class 2 Type V CRISPR-Cpf1 systems, modifications of Cpf1 proteins, variants of Cpf1 proteins, Cpf1 orthologs, and combinations thereof. “dCpf1,” as used herein, refers to variants of Cpf1 protein that are nuclease-deactivated Cpf1 proteins, also termed “catalytically inactive Cpf1 protein,” or “enzymatically inactive Cpf1.” Cpf1 proteins are known, for example, Francisella tularensis (UniProtKB—AOQ7Q2 (CPF1_FRATN)), and Acidaminococcus sp. (UniProtKB—U2UMQ6 (CPF1_ACISB)).

As used herein, a “guide” refers to any polynucleotide that site-specifically guides a Cas protein to a target nucleic acid sequence. In a preferred embodiment, a guide is capable of forming a complex with a Class 2 CRISPR-associated protein, for example a Class 2 Type II CRISPR-associated protein (e.g., a Cas9 protein or a dCas9 protein) or a Class 2 Type V CRISPR-associated protein, (e.g., a Cpf1 protein or a dCpf1 protein). Many such guides are known, including but not limited to single-guide (sg) RNA (including miniature and truncated sgRNAs), dual-guide (dg) RNA (including but not limited to crRNA/tracrRNA molecules), and the like. In some embodiments, a guide comprises RNA, DNA, or combinations of RNA and DNA. As used herein, a “locus-specific guide” refers to a guide polynucleotide that contains a spacer sequence complementary to a target nucleic acid sequence within a selected locus (e.g., sgRNA_{target}, sgRNA_{K}_{ᵤ}). A target nucleic acid sequence can, for example, be in a locus to be modified by incorporation of a donor polynucleotide (or portion or copy thereof). The locus-specific guide can associate with a Cas protein (e.g., a Cas9 protein or a Cpf1 protein) to target nucleic acid sequences in a cell (e.g., genomic DNA) for binding or cleavage. As used herein, a “locus-specific guide polynucleotide” typically refers to a polynucleotide that encodes a locus-specific guide RNA. A “Cas-specific guide” (e.g., a Cas9-specific guide or a Cpf1-specific guide) refers to a guide that contains a spacer sequence complementary to a sequence in a polynucleotide encoding a Cas protein. The Cas-specific guide can associate with its cognate Cas protein to target a polynucleotide encoding the Cas protein to bind or cleave the polynucleotide. For example, as described herein, a Cas9-specific guide RNA/Cas9 protein complex can “turn off” cleavage by a locus-specific guide RNA/Cas9 protein complex by cleaving the coding sequence for the Cas9 protein (thus stopping production of the Cas9 protein by terminating transcription of the Cas9 coding sequence). Similarly, a Cpf1-specific guide RNA/Cpf1 protein complex can “turn off” cleavage by a locus-specific guide RNA/Cpf1 protein complex by cleaving the coding sequence for the Cpf1 protein (thus stopping production of the Cpf1 protein by terminating transcription of the Cpf1 coding sequence). A “Cas-specific guide polynucleotide” typically refers to a polynucleotide that encodes a Cas-specific guide RNA.

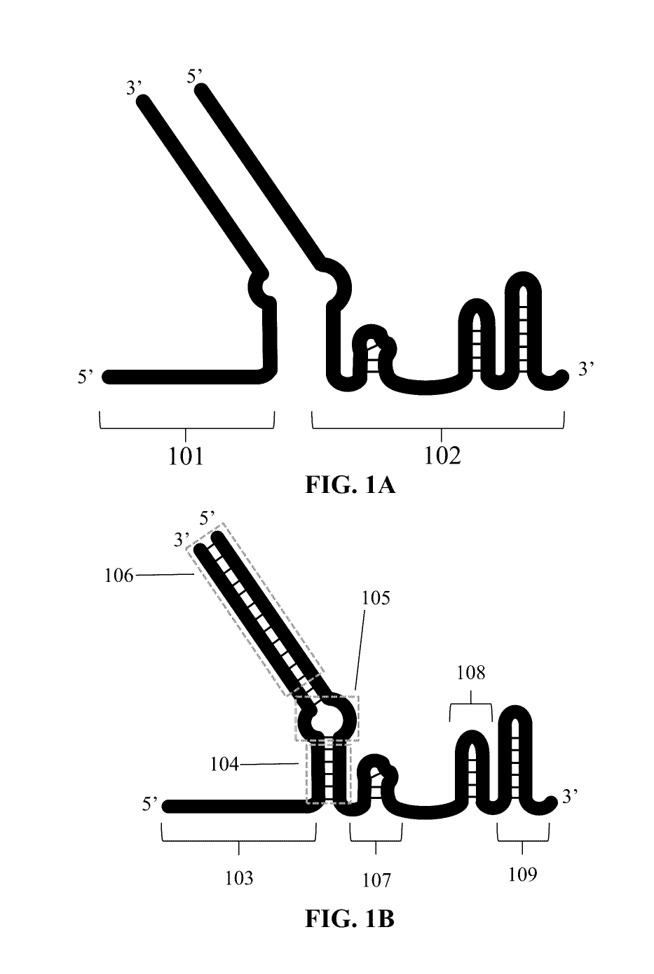

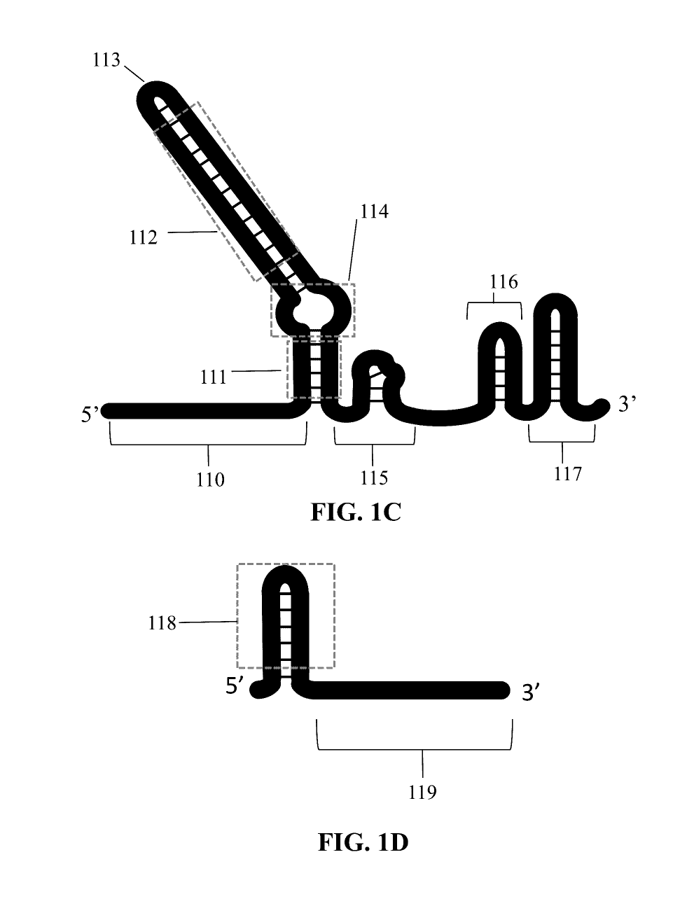

As used herein, “dual-guide RNA” typically refers to a two-component RNA system for a polynucleotide component capable of associating with a cognate Cas9 protein. FIG. 1A shows a two-RNA component (dual-guide RNA (dgRNA)) Class 2 Type II CRISPR-Cas9 system comprising a crRNA (FIG. 1A, 101) and a tracrRNA (FIG. 1A, 102). FIG. 1B illustrates the formation of base-pair hydrogen bonds between the crRNA and the tracrRNA to form secondary structure (see, e.g., U.S. Published Patent Application No. 2014-0068797, published 6 Mar. 2014; see also Jinek, M., et al., Science 337:816-21(2012)). FIG. 1B presents an overview of and nomenclature for secondary structural elements of the crRNA and tracrRNA of Streptococcus pyogenes Cas9, including the following: a spacer element (i.e., a target nucleic acid binding sequence) (FIG. 1B, 103); a first stem element comprising a lower stem element (FIG. 1B, 104), a bulge element comprising unpaired nucleotides (FIG. 1B, 105), and an upper stem element (FIG. 1B, 106); a nexus element (FIG. 1B, 107); a second hairpin element comprising a second stem element (FIG. 1B, 108); and a third hairpin element comprising a third stem element (FIG. 1B, 109) (see, e.g., U.S. Published Patent Application No. 2014-0068797, published 6 Mar. 2014; see also Jinek, M., et al., Science 337:816-21(2012)). A dual-guide RNA is capable of forming a nucleoprotein complex with a cognate Cas9 protein, wherein the complex is capable of targeting a target nucleic acid sequence complementary to the spacer sequence.

As used herein, “single-guide RNA” (sgRNA) typically refers to a one-component RNA system for a polynucleotide component capable of associating with a cognate Cas9 protein. FIG. 1C illustrates a single-guide RNA (sgRNA) wherein the crRNA is covalently joined to the tracrRNA and forms a RNA polynucleotide secondary structure through base-pair hydrogen bonding (see, e.g., U.S. Published Patent Application No. 2014-0068797, published 6 Mar. 2014). The figure presents an overview of and nomenclature for secondary structural elements of an sgRNA of Streptococcus pyogenes Cas9, including the following: a spacer element (FIG. 1C, 110); a first stem element comprising a lower stem element (FIG. 1C, 111), a bulge element comprising unpaired nucleotides (FIG. 1C, 114), and an upper stem element (FIG. 1C, 112); a loop element (FIG. 1C, 113) comprising unpaired nucleotides; a nexus element (FIG. 1C, 115); a second hairpin element comprising a second stem element (FIG. 1C, 116); and a third hairpin element comprising a third stem element (FIG. 1C, 117) (see, e.g., Figures 1 and 3 of Briner, A. E., et al., Molecular Cell Volume 56(2):333-339 (2014)). An sgRNA is capable of forming a nucleoprotein complex with a cognate Cas9 protein, wherein the complex is capable of targeting a target nucleic acid sequence complementary to the spacer sequence.

“Guide crRNA,” as used herein, typically refers to a one-component RNA system for a polynucleotide component capable of associating with a cognate Cpf1 protein. FIG. 1D presents an example of a Class 2 Type V CRISPR-Cpf1-associated RNA (Cpf1-crRNA) (see, e.g., Zetsche, B., et al., Cell 163:1-13 (2015)). FIG. 1D shows a one-RNA component Class 2 Type V CRISPR-Cpf1 system, such as is present in Acidominococcus and Lachnospiraceae, comprising a crRNA having a stem-loop element (FIG. 1D, 118) and a spacer element (FIG. 1D, 119). A guide crRNA is capable of forming a nucleoprotein complex with a cognate Cpf1 protein, wherein the complex is capable of targeting a target nucleic acid sequence complementary to the spacer sequence.

As used herein, “cognate” typically refers to a Cas protein and a guide that are capable of forming a nucleoprotein complex capable of directed binding to a target nucleic acid complementary to a target nucleic acid binding sequence present in the guide.

As used herein, “complementarity” refers to the ability of a nucleic acid sequence to form hydrogen bond(s) with another nucleic acid sequence (e.g., through traditional Watson-Crick base-pairing). A percent complementarity indicates the percentage of residues in a nucleic acid molecule that can form hydrogen bonds with a second nucleic acid sequence. When two polynucleotide sequences have 100% complementary, the two sequences are perfectly complementary, i.e., all of the contiguous residues of a first polynucleotide hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, and between a protein and a protein). Such non-covalent interaction is also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as contact between a protein and the phosphate residues in a DNA backbone. Binding interactions can be characterized by a dissociation constant (K_{d}). “Affinity” refers to the strength of binding. An increased binding affinity is correlated with a lower K_{d}. An example of non-covalent binding is hydrogen bond formation between base pairs.

As used herein, a Cas protein (e.g., a Cas9 protein or Cpf1 protein) is said to “target” a polynucleotide if a Cas protein/guide nucleoprotein complex binds or cleaves a polynucleotide at the target nucleic acid sequence within the polynucleotide.

As used herein, “double-strand break” (DSB) refers to both strands of a double-stranded segment of DNA being severed. In some instances, when such a break occurs, one strand can be said to have a “sticky end” where nucleotides are exposed and not hydrogen bonded to nucleotides on the other strand. In other instances, a “blunt end” can occur where both strands remain fully base-paired with each other despite the DSB.

As used herein, a “donor polynucleotide” can be a double-strand polynucleotide (e.g., DNA), a single-stranded polynucleotide (e.g., DNA oligonucleotides), or a combination thereof. Donor polynucleotides comprise homology arms flanking the insertion sequence (e.g., DSBs in the DNA). The homology arms on each side can vary in length. Parameters for the design and construction of donor polynucleotides are well-known in the art (see, e.g., Ran, F., et al., Nat Protoc. 8(11):2281-2308 (2013); Smithies, O., et al., Nature 317:230-234 (1985); Thomas, K., et al., Cell 44:419-428 (1986); Wu, S., et al., Nat. Protoc. 3:1056-1076 (2008); Singer, B., et al., Cell 31:25-33 (1982); Shen, P., et al., Genetics 112:441-457 (1986); Watt, V., et al., Proc. Natl. Acad. Sci. USA 82:4768-4772 (1985), Sugawara, N., et al., Mol Cell Biol 12(2):563-575 (1992); Rubnitz, J., et al., Mol Cell Biol 4(11):2253-2258 (1984); Ayares, D., et al., Proc. Natl. Acad. Sci. USA 83(14):5199-5203 (1986); Liskay, R, et al., Genetics 115(1):161-167 (1987)).

As used herein, “homology-directed repair” (HDR) refers to DNA repair that takes place in cells, for example, during repair of a DSB in DNA. HDR requires nucleotide sequence homology and uses a donor polynucleotide to repair the sequence where the DSB (e.g., within a target DNA sequence) occurred. The donor polynucleotide generally has the requisite sequence homology with the sequence flanking the DSB so that the donor polynucleotide can serve as a suitable template for repair. HDR results in the transfer of genetic information from, for example, the donor polynucleotide to the DNA target sequence. HDR may result in alteration of the DNA target sequence (e.g., insertion, deletion, mutation) if the donor polynucleotide sequence differs from the DNA target sequence and part or all of the donor polynucleotide is incorporated into the DNA target sequence. In some embodiments, an entire donor polynucleotide, a portion of the donor polynucleotide, or a copy of the donor polynucleotide is integrated at the site of the DNA target sequence. HDR is understood to be mostly active during the S and G₂ phases of the cell cycle (see, e.g., Lin, et al, eLife e04766. DOI: 10.7554/eLife.04766 (2014); Aylon, Y., et al., EMBO J. 23:4868-4875 (2004); Ira, G., et al., Nature 431:1011-1017 (2004); Huertas, P., et al., Nature 455:689-692 (2008); Huertas, P., et al., J. Biol. Chem. 284:9558-9565 (2009)).

A “genomic region” is a segment of a chromosome in the genome of a host cell that is present on either side of the target nucleic acid sequence site or, alternatively, also includes a portion of the target site. The homology arms of the donor polynucleotide have sufficient homology to undergo homologous recombination with the corresponding genomic regions.

In some embodiments, the homology arms of the donor polynucleotide share significant sequence homology to the genomic region immediately flanking the target site; it is recognized that the homology arms can be designed to have sufficient homology to genomic regions farther from the target site.

As used herein, “non-homologous end joining” (NHEJ) refers to the repair of a DSB in DNA by direct ligation of one end of the break to the other end of the break without a requirement for a donor polynucleotide. NHEJ is a DNA repair pathway available to cells to repair DNA without the use of a repair template. NHEJ in the absence of a donor polynucleotide often results in nucleotides being randomly inserted or deleted at the site of the DSB. NHEJ dominates DNA repair during the G₁, G₀, and M phases of the cell cycle (see, e.g., Lin, et al., eLife e04766. DOI: 10.7554/eLife.04766 (2014); Aylon, Y., et al., EMBO J. 23:4868-4875 (2004); Ira, G., et al., Nature 431:1011-1017 (2004); Huertas, P., et al., Nature 455:689-692 (2008); Huertas, P., et al., J. Biol. Chem. 284:9558-9565 (2009)). The initial step in NHEJ is typically the recognition of a DSB by a Ku heterodimer composed of Ku70 and Ku80. The Ku heterodimer serves as a scaffold that recruits other proteins involved in the NHEJ pathway. Following recruitment of these other factors, the DNA ends often undergo resection, or trimming, of nucleotides. In other cases, polymerases may add nucleotides to the DNA ends. Following this end processing, the two ends are ligated back together.

As used herein, a “protein that drives the NHEJ pathway” refers to any protein that contributes to NHEJ, whether directly or indirectly. Examples include, but are not limited to, Ku70, Ku80, DNA-dependent protein kinase, catalytic subunit (DNA-PKcs), DNA Ligase IV, X-ray repair cross-complementing protein 4 (XRCC4), XRCC4-like factor (XLF), Artemis, DNA polymerase mu, DNA polymerase lambda, bifunctional polynucleotide phosphatase/kinase (PNKP), Aprataxin, Aprataxin polynucleotide kinase/phosphatase-like factor (APLF), and the like, and orthologs thereof.

As used herein, a “NHEJ pathway-specific regulatory element” refers to a regulatory element that drives expression of a protein that drives the NHEJ pathway, such as, for example, a promoter or enhancer derived from a gene encoding such protein (e.g., a Ku protein).

As used herein, a “NHEJ pathway-specific guide” refers to a guide (e.g., a guide RNA) that contains a spacer sequence complementary to a sequence in a gene that encodes a protein that drives the NHEJ pathway. This guide (e.g., a guide RNA) can associate with a dCas protein (e.g., a dCas9 protein or dCpf1 protein) to form a complex that is capable of binding the gene.

As used herein, a “NHEJ pathway-specific guide polynucleotide” refers to a polynucleotide that encodes a NHEJ pathway-specific guide RNA.

“Ku protein” refers to a Ku70 protein, a Ku80 protein, and orthologs thereof.

“Microhomology-mediated end joining” (MMEJ) is pathway for repairing a DSB in DNA. MMEJ is associated with deletions flanking a DSB and involves alignment of microhomologous sequences internal to the broken ends before joining. MMEJ is genetically defined and requires the activity of, for example, CtIP, Poly(ADP-Ribose) Polymerase 1 (PARP1), DNA polymerase theta (Pol θ), DNA Ligase 1 (Lig 1), DNA Ligase 3 (Lig 3). Additional genetic components are known in the art (see, e.g., Sfeir, A., et al., Trends Biochem Sci. 40:701-714 (2015)).

As used herein, “DNA repair” encompasses any process whereby cellular machinery repairs damage to a DNA molecule contained in the cell. The damage repaired can include single-strand breaks or double-strand breaks. At least three mechanisms exist to repair DSBs: HDR, NHEJ, and MMEJ. “DNA repair” is also used herein to refer to DNA repair resulting from human manipulation, wherein a target locus is modified, e.g., by inserting, deleting, substituting nucleotides, all of which represent forms of genome editing.

As used herein, “recombination” refers to a process of exchange of genetic information between two polynucleotides.

As used herein, “cell state” refers to any specific condition of a cell. This condition can be, for example, a specific metabolic state or the state of a cell in relation to the cell cycle (e.g., cell cycle phase). Cell state can also refer to a stage of differentiation, e.g., ranging from undifferentiated to fully differentiated. In some cases, a particular cell state can be initiated by some exogenous stimulus. A change in cell state can be accompanied by differential expression of specific genes relative to the previous cell state (e.g., differential gene expression may be the cause or result of the change in state).

As used herein, “cell cycle” refers to the progression of events that take place in a cell that lead to its division and duplication. In prokaryotic cells, this process is termed “binary fission.” In eukaryotes, the cell cycle can be divided into several phases. These phases are G₁ (Gap 1, preparation for DNA synthesis), S (Synthesis, DNA replication), G₂ (Gap 2, preparation for cell division), M (Mitosis, cell division) and G₀ (Gap 0, resting). It has been shown that regulatory proteins called cyclins and cyclin-dependent kinases regulate the progression of a eukaryotic cell through the cell cycle. Additionally, other proteins and transcription factors have been shown to be expressed in specific phases of the cell cycle. In particular, expression of proteins that are part of the HDR pathway are expressed during the S and G₂ phases of the cell cycle.

“Regulatory element” and “regulatory sequences,” as used herein, are interchangeable and include promoters, enhancers, internal ribosome entry sites (IRES), and other expression control elements (e.g., transcription start sites; and transcription termination signals, such as polyadenylation signals and poly-U sequences). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al, Cell 41:521-530 (1985)), the SV40 promoter, the dihydrofolate reductase promoter, the β-actin promoter, the phosphoglycerol kinase (PGK) promoter, and the EF1α promoter.

Also encompassed by the term “regulatory element” are repressor domains such as the KRAB domain. As described by Lupo, A., et al, Curr Genomics 14(4): 268-278 (2013), the KRAB domain is a potent transcriptional repression module and is located in the amino-terminal sequence of most C2H2 zinc finger proteins (Margolin, J., et al, Proc. Natl. Acad. Sci. 91:4509-4513 (1994); Witzgall, R., et al, Proc. Natl. Acad. Sci. 91:4514-4518 (1994)). The KRAB domain typically binds to co-repressor proteins and/or transcription factors via protein-protein interactions, causing transcriptional repression of genes to which KRAB zinc finger proteins (KRAB-ZFPs) bind (Friedman J R, Fredericks W J, Jensen, D., et al, Genes Dev. 10:2067-2678 (1996)). An example of one such gene to which KRAB-ZFPs bind is the Ku gene. In humans, KRAB-ZPFs constitute one of the largest families of transcriptional regulators. Due to the presence of the KRAB domain, which is a powerful transcriptional repressor domain, most members of the KRAB-ZFPs family have a role in regulating embryonic development, cell differentiation, cell proliferation, apoptosis, neoplastic transformation and cell cycle regulation (see, e.g., Urrutia, R., Genome Biol. 4:231-238 (2003)). Also encompassed by the term “regulatory element” are enhancer elements, such as WPRE; CMV enhancers; the R-U5′ segment in LTR of HTLV-I (see, e.g., Takebe, Y., et al, Mol. Cell. Biol. 8(1):466-472 (1988)); SV40 enhancer; and the intron sequence between exons 2 and 3 of rabbit β-globin (see, e.g., O'Hare, K., Proc. Natl. Acad. Sci. USA 78(3):1527-1531 (1981)). It will be appreciated by those skilled in the art that the design of an expression vector can depend on such factors as the choice of the host cell to be transformed, the level of expression desired, and the like. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, and the like).

A regulatory element is “active in response to a cell state” when the activity of the regulatory element is modulated by the cell state. The cell state may be one that increases or decreases activity of the regulatory element. In cases where the cell state is characterized by the level of protein, the relationship between the protein level and activity of the regulatory element can be direct or inverse. If activity of the regulatory element increases when the protein level increases, and vice versa, the relationship is direct. If activity of the regulatory element decreases when the protein level increases, and vice versa, the relationship is inverse.

“Gene,” as used herein, refers to a polynucleotide sequence comprising exon(s) and any associated regulatory sequences. A gene may further comprise intron(s) and/or untranslated region(s) (UTR).

As used herein, “level” includes presence, absence, or an amount.

“Operably linked,” as used herein, refers to polynucleotide sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function when separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product(s).” Expression may include splicing the mRNA in a eukaryotic cell, if the polynucleotide is derived from genomic DNA.

“Vector” and “plasmid,” as used herein, refer to a polynucleotide vehicle to introduce genetic material into a cell. Vectors can be linear or circular. Vectors can contain a replication sequence capable of effecting replication of the vector in a suitable host cell (i.e., an origin of replication). Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art. The four major types of vectors are plasmids, viral vectors, cosmids, and artificial chromosomes. Typically, vectors comprise an origin of replication, a multicloning site, and/or a selectable marker. An expression vector typically comprises an expression cassette.

As used herein, “expression cassette” refers to a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, a “targeting vector” is a recombinant DNA construct typically comprising tailored DNA arms, homologous to genomic DNA, that flank elements of a target gene or target sequence (e.g., a DSB). A targeting vector comprises a donor polynucleotide. Elements of the target gene can be modified in a number of ways including deletions and/or insertions. A defective target gene can be replaced by a functional target gene, or in the alternative a functional gene can be knocked out. Optionally, the donor polynucleotide of a targeting vector comprises a selection cassette comprising a selectable marker that is introduced into the target gene. Targeting regions adjacent or within a target gene can be used to affect regulation of gene expression.

As used herein, a “transcript separator sequence” refers to a sequence in an RNA transcript that liberates two RNA species in the transcript from one another. The transcript separator sequence can be disposed between the two RNA species and can, for example, be a self-cleaving ribozyme or a sequence recognized by a ribonuclease (e.g., Csy4).

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary structure and three-dimensional structure. The terms encompass known analogs of natural nucleotides and nucleotides that are modified in the base, sugar and/or phosphate moieties. Analogs of a particular nucleotide have the same base-pairing specificity (e.g., an analog of A base-pairs with T). A polynucleotide may comprise one modified nucleotide or multiple modified nucleotides. Examples of modified nucleotides include methylated nucleotides. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages, that are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, Mass.) nucleosides, glycol nucleic acid, bridged nucleic acids, and morpholino structures.

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at the 5′ or 3′ end of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). Typically, such polynucleotides have phosphoramidite groups on the 5′-OH position and a dimethoxytrityl (DMT) protecting group on the 3′-OH position. Normally, the DMT protecting group is on the 5′-OH and the phosphoramidite is on the 3′-OH. The most common use of linkage inversion is to add a 3′-3′ linkage to the end of a polynucleotide with a phosphorothioate backbone. The 3′-3′ linkage stabilizes the polynucleotide to exonuclease degradation by creating an oligonucleotide having two 5′-OH ends and no 3′-OH end.

Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity, as used herein, between two polynucleotides or two polypeptides is typically between about 90% identity and 100% identity, for example, about 90% identity or higher, preferably about 95% identity or higher, more preferably about 98% identity or higher. A moderate degree of sequence identity, as used herein, between two polynucleotides or two polypeptides is typically between about 80% identity to about 85% identity, for example, about 80% identity or higher, preferably about 85% identity. A low degree of sequence identity, as used herein, between two polynucleotides or two polypeptides is typically between about 50% identity and 75% identity, for example, about 50% identity, preferably about 60% identity, more preferably about 75% identity. For example, a Cas protein (e.g., a Cas9 comprising amino acid substitutions or a Cpf1 comprising amino acid substitutions) can have a moderate degree of sequence identity, or preferably a high degree of sequence identity, over its length to a reference Cas protein (e.g., a wild-type Cas9 or a wild-type Cpf1, respectively). As another example, a guide can have a moderate degree of sequence identity, or preferably a high degree of sequence identity, over its length compared to a reference wild-type polynucleotide that complexes with the reference Cas protein (e.g., an sgRNA that forms a complex with Cas9 or a crRNA that forms a a complex with Cpf1).

As used herein, “hybridization” or “hybridize” or “hybridizing” is the process of combining two complementary single-stranded DNA or RNA molecules and allowing them to form a single double-stranded molecule (DNA/DNA, DNA/RNA, RNA/RNA) through hydrogen base-pairing. Hybridization stringency is typically determined by the hybridization temperature and the salt concentration of the hybridization buffer, for example, high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; low stringency, approximately 0.33M to approximately 0.82M salt, hybridization temperature 40° C. to 48° C. below Tₘ. Tₘ, of duplex nucleic acids is calculated by standard methods well-known in the art (Maniatis, T., et al Molecular Cloning: A Laboratory Manual, Cold Spring Harbor Laboratory Press: New York (1982); Casey, J., et al., Nucleic Acids Res., 4:1539-1552 (1977); Bodkin, D. K., et al., J. Virol. Methods, 10(1):45-52 (1985); Wallace, R. B., et al., Nucleic Acids Res. 9(4):879-894 (1981)). Algorithm prediction tools to estimate Tₘ, are also widely available. High stringency conditions for hybridization typically refer to conditions under which a nucleic acid having complementarity to a target sequence predominantly hybridizes with the target sequence, and substantially does not hybridize to non-target sequences. Typically, hybridization conditions are of moderate stringency, preferably high stringency.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts discussed above). Furthermore, essentially any polypeptide or polynucleotide is available from commercial sources.

The terms “fusion protein” and “chimeric protein,” as used herein, refer to a single protein created by joining two or more proteins, protein domains, or protein fragments that do not naturally occur together in a single protein. For example, a fusion protein can contain a first domain from a Cas9 or a Cpf1 protein and a second domain from a protein other than a Cas9 protein or a Cpf1 protein. The modification of a polypeptide to include such domains in a fusion protein may confer additional activity to the modified polypeptide. Such activities can include nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity) that modifies a polypeptide associated with target nucleic acid sequence (e.g., a histone). A fusion protein can also comprise epitope tags (e.g., histidine tags, FLAG® (Sigma Aldrich, St. Louis, Mo.) tags, Myc tags), reporter protein sequences (e.g., glutathione-S-transferase, beta-galactosidase, luciferase, green fluorescent protein, cyan fluorescent protein, yellow fluorescent protein), nucleic acid binding domains (e.g., a DNA binding domain, an RNA binding domain). In some embodiments, linker sequences are used to connect the two or more proteins, protein domains, or protein fragments.

As used herein, a “repressor protein” refers to a protein that binds to a repressor binding sequence in DNA and inhibits transcription of a linked gene. The lac repressor protein is a prototypical DNA-binding repressor that inhibits the expression of lac genes coding for proteins involved in the metabolism of lactose in bacteria. As used herein, a “repressor moiety” refers to a portion of a larger molecule (typically a repressor protein) that represses transcription when the repressor moiety is targeted to a suitable region in a gene, such as an enhancer domain. The repressor moiety can perform this function as part of a fusion protein, e.g., with a targeting moiety, such as an inactive Cas protein (e.g., dCas9 or dCpf1).

As used herein, a “repressor polynucleotide” refers to a polynucleotide that encodes a repressor protein or a repressor moiety.

A “lacO operator sequence” refers to a DNA sequence that lies partially within the lacP promoter sequence and that is a repressor binding sequence for the lac repressor protein.

A “lacI sequence” or “lacI gene” refers to a DNA sequence that encodes the lac protein repressor.

As used herein, a “host cell” generally refers to a biological cell. A cell can be the basic structural, functional and/or biological unit of a living organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoal cell, a cell from a plant (e.g., cells from plant crops, fruits, vegetables, grains, soy bean, corn, maize, wheat, seeds, tomatoes, rice, oil-producing Brassica (for example but not limited to oil seed rape/canola), cassava, sunflower, sorghum, millet, alfalfa, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. agardh, and the like), seaweeds (e.g, kelp), a fungal cell (e.g, a yeast cell, a cell from a mushroom), an animal cell, a cell from an invertebrate animal (e.g., fruit fly, cnidarian, echinoderm, nematode, and the like), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, and the like). Further, a cell can be a stem cell or a progenitor cell.

As used herein, “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells can be embryonic, fetal, amniotic, adult, or induced pluripotent stem cells.

As used herein, “induced pluripotent stem cells” refers to a type of pluripotent stem cell that is artificially derived from a non-pluripotent cell, typically an adult somatic cell, by inducing expression of specific genes.

“Plant,” as used herein, refers to whole plants, plant organs, plant tissues, germplasm, seeds, plant cells, and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells and culture (e.g., single cells, protoplasts, embryos, and callus tissue). The plant tissue may be in plant or in a plant organ, tissue or cell culture. “Plant organ” refers to plant tissue or a group of tissues that constitute a morphologically and functionally distinct part of a plant.

“Subject,” as used herein, refers to any member of the phylum Chordata, including, without limitation, humans and other primates, including non-human primates such as rhesus macaque, chimpanzees and other apes and monkey species; farm animals, such as cattle, sheep, pigs, goats and horses; domestic mammals, such as dogs and cats; laboratory animals, including rabbits, mice, rats and guinea pigs; birds, including domestic, wild, and game birds, such as chickens, turkeys and other gallinaceous birds, ducks, and geese; and the like. The term does not denote a particular age or gender. Thus, adult, young, and newborn individuals are intended to be covered as well as male and female. In some embodiments, a host cell is derived from a subject (e.g., stem cells, progenitor cells, tissue specific cells). In some embodiments, the subject is a non-human subject.

The terms “wild-type,” “naturally occurring,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the terms “engineered,” “genetically engineered,” “recombinant,” “modified,” and “non-naturally occurring” are interchangeable and indicate intentional human manipulation.

As used herein, “transgenic organism” refers to an organism whose genome includes a recombinantly introduced polynucleotide. The term includes the progeny (any generation) of a directly created transgenic organism, provided that the progeny has the recombinantly introduced polynucleotide.

As used herein, “isolated” can refer to a nucleic acid or polypeptide that, by the human intervention, exists apart from its native environment and is therefore not a product of nature. An isolated nucleic acid or polypeptide can exist in a purified form and/or can exist in a non-native environment such as, for example, in a recombinant cell.

The engineered Class 2 CRISPR-Cas systems described herein are based on components from Class 2 CRISPR-Cas systems (e.g., Type II CRISPR-Cas9 systems and Type V CRISPR-Cpf1 systems). The engineered Class 2 CRISPR-Cas systems are used to bind or cleave target nucleic acid sequences in a directed manner, wherein expression of the engineered Class 2 CRISPR-Cas system components is conditional and dependent on a cell state of a host cell. In a general aspect, the present invention relates to a cell cycle regulated expression of a Cas protein and/or cognate guide RNA coding sequences.

For genetic engineering of cells and organisms, it is desirable to improve the frequency of HDR-mediated polynucleotide integration. In preferred embodiments, the engineered Class 2 CRISPR-Cas systems described herein are designed to provide improvement of the frequency of HDR-mediated integration of a polynucleotide into a target nucleic acid in a host cell by using conditional expression (e.g., cell-state mediated regulation) of components from the engineered Class 2 CRISPR-Cas systems. The improvement of frequency of HDR-mediated integration is relative to HDR frequencies seen in the same cells and organisms in the absence of the conditional expression (e.g., cell-state mediated regulation) of components from the engineered Class 2 CRISPR-Cas systems (e.g., compared to the frequency of HDR in the wild-type host cell or organism).

As described herein, the present invention includes modulation of Cas protein expression in response to cell states (e.g., cell cycle phases). Accordingly, the Class 2 CRISPR-Cas systems of the present invention include polynucleotide compositions comprising a polynucleotides encoding a Cas protein, wherein the polynucleotide is operably linked to a regulatory element that is active in response to a cell state of a host cell (e.g., a eukaryotic host cell). In some embodiments, polynucleotide compositions also include polynucleotides encoding guide RNAs. In some embodiments, targeting vectors comprise donor polynucleotides.

Examples of natural cell states that are conducive to HDR are G₂ and S phases of the cell cycle. Accordingly, regulatory elements active in G₂ or S phase can be operably linked to Cas protein coding sequences and/or cognate guide coding sequences. In some embodiments to promote HDR, the guide has homology to a target site, and a donor polynucleotide having homology to sequences flanking the target site is provided.

The cell state can be natural or induced by human action, but is usually transient. Natural or engineered cell states include phases of the cell cycle (e.g., S phase), as well as exogenous stimuli (e.g., lipopolysaccharide, auxin).

Examples of cell state (e.g., cell cycle) regulatory elements useful in embodiments of the invention include, but are not limited to, transcriptional regulatory elements associated with expression of the following proteins: CDK1, maximally expressed in G₂ (SEQ ID NO:1; Badie, C., et al., Molecular and Cellular Biology 20(7):2358-2366 (2000)); Cyclin A, maximally expressed in G₂/S (SEQ ID NO:2; Badie, C., et al., Molecular and Cellular Biology 20(7):2358-2366 (2000)); Cyclin B1, maximally expressed in G₂/M (SEQ ID NO:3; Hwang, A., et al., Journal of Biological Chemistry 273(47):31505-31509 (1998); and the family of E2F transcription factors, predominantly active in G₁/S (Dimova, D., et al., Oncogene 24:2810-2826 (2005); Dyson, N., Genes Dev. 12: 2245-2262 (1998); Helin, K., Curr. Opin. Gene Dev. 8:28-35 (1998); Nevins, J., Cell Growth Differ. 9:585-59 (1998); DeGregori, J., Biochim. Biophys. Acta 1602:131-150 (2002); Trimarchi, J., et al., Nat. Rev. Mol. Cell. Biol. 3:11-20 (2002); Rabinovich, A., Genome Res. 18(11):1763-1777 (2008)).

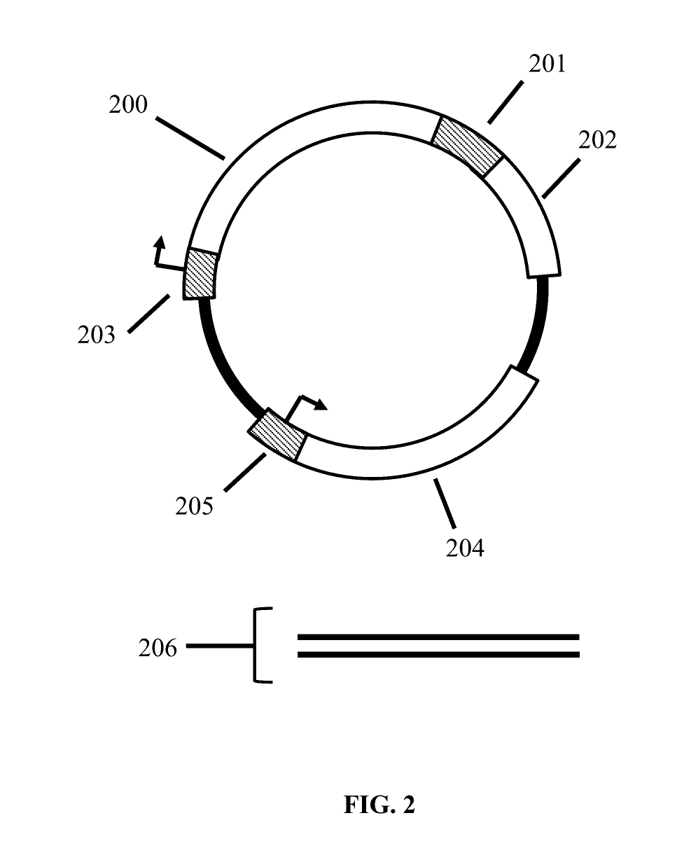

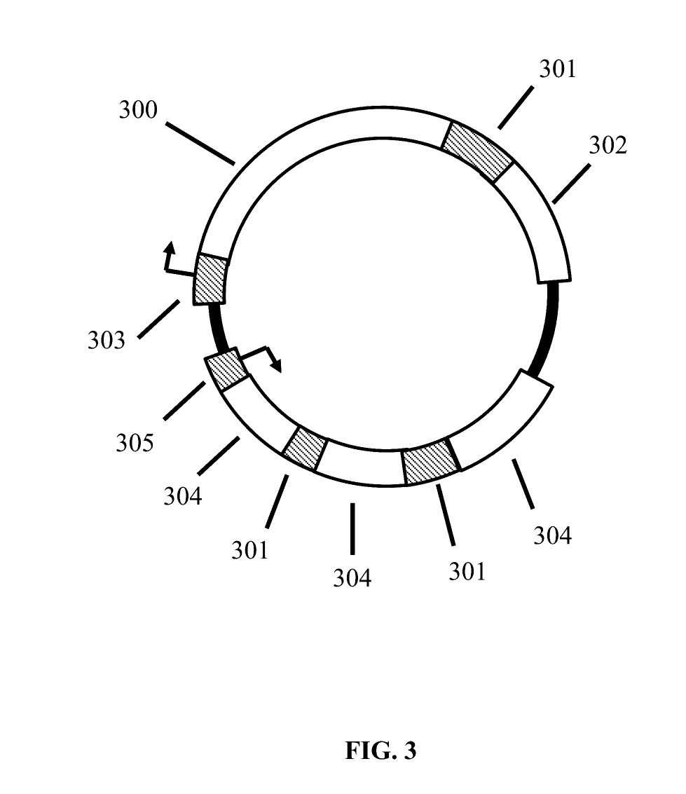

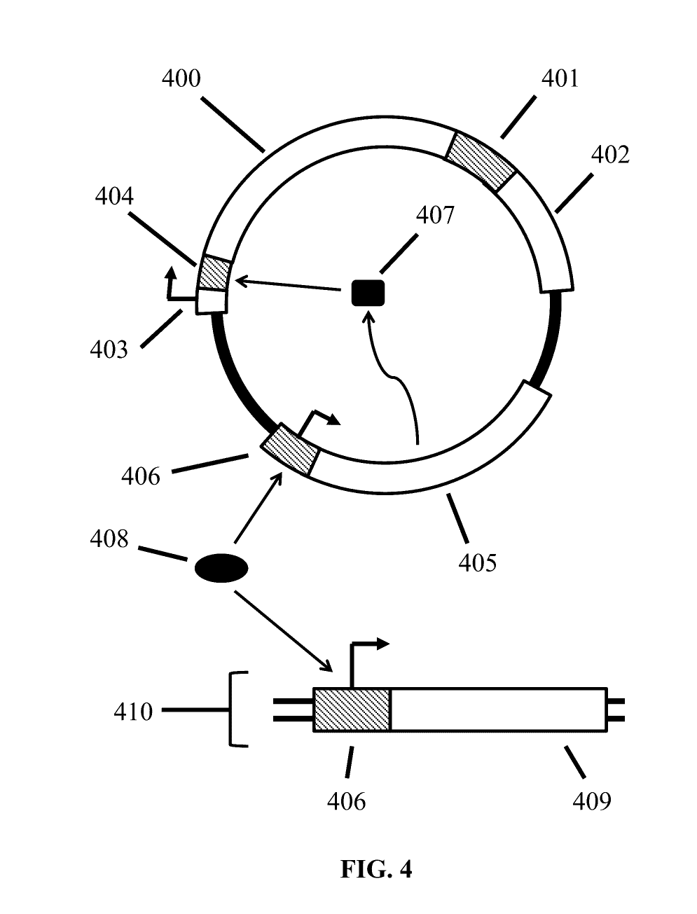

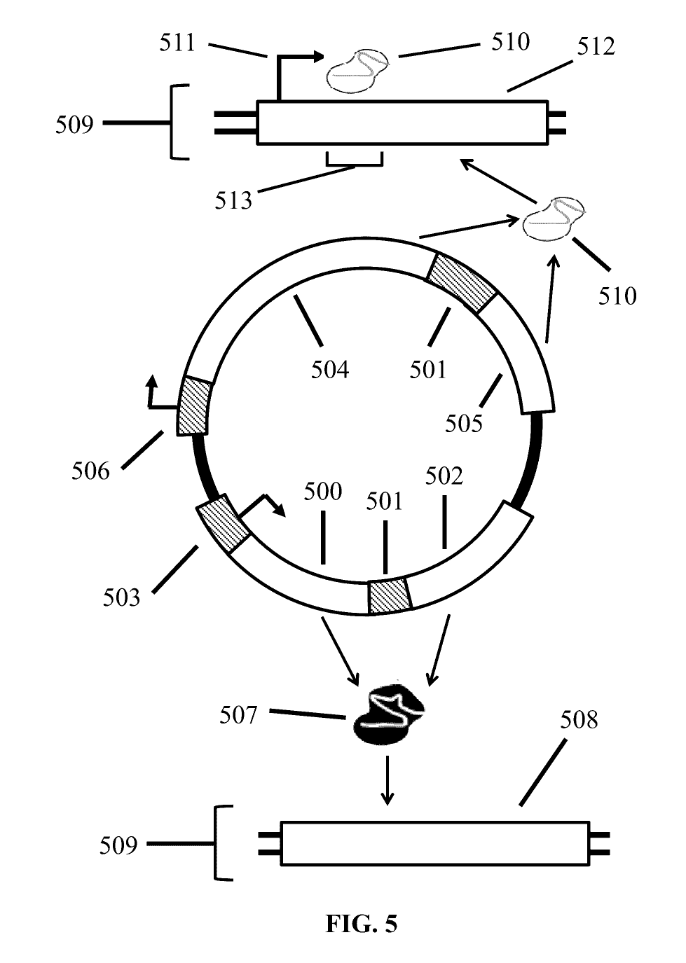

Engineered Class 2 CRISPR-Cas systems described herein can be used for enhancing directed DNA repair. The Cas protein is conditionally expressed. This conditional expression occurs in response to a specific cellular state, such as a cell cycle phase. In preferred embodiments to promote HDR, regulatory elements to conditionally express the Cas protein are associated with regulatory elements from genes actively expressed in S phase or G₂ phase. This is exemplified in FIG. 2 using an engineered Class 2 Type II CRISPR-Cas9 system. The system contains the cas9 gene (FIG. 2, 200), which is a polynucleotide encoding an RNA that is translated into the Cas9 protein. The system also contains an sgRNA (FIG. 2, 202; “sgRNA_{target}”) capable of targeting a genomic region, as well as a transcript separator sequence (FIG. 2, 201) that liberates the cas9 RNA from the sgRNA_{target}. In some embodiments, this transcript separator can be a self-cleaving ribozyme, such as a hammerhead ribozyme. In other embodiments, the transcript separator can be an RNA sequence recognized by a ribonuclease such as Csy4. The Cas9-transcript separator-sgRNA_{target} polynucleotide is operably linked to Promoter A (FIG. 2, 203; the direction of transcription is indicated by an arrow). Alternatively, the sgRNA_{target} can be expressed as a different transcript that is also operably linked to another copy of Promoter A. The system can also contain a polynucleotide encoding an sgRNA that targets to the cas9 gene (FIG. 2, 204, “sgRNA_{Cas9}”). This transcript is operably linked to Promoter B (FIG. 2, 205; the direction of transcription is indicated by an arrow). The vector backbone is shown as a solid black curve.

Embodiments of this system include Promoter A comprising a first regulatory element that is active in response to a first cell cycle phase of the host cell (e.g., a regulatory element active in S and/or G₂ when expression of proteins that are part of the HDR pathway are expressed) and Promoter B comprising a second regulatory element that is active in response to a second cell cycle phase of the host cell (e.g., a regulatory element active in G₁, M, and/or G₀ when proteins that are part of the HDR pathway are present at lower levels than S and/or G₂, i.e., the NHEJ pathway is more active than the HDR pathway). This combination of regulatory elements allows for expression of the Cas protein (e.g., a Cas9 protein or a Cpf1 protein) and the sgRNA_{target} when the HDR pathway is more active and HDR occurs with greater efficiency. When the sgRNA_{Cas9} is expressed, it can form a complex with the Cas protein and cleave the Cas protein coding sequence to terminate expression of Cas protein.

In some embodiments, this system can be transfected into cells with one or more oligonucleotides (FIG. 2, 206), such as a donor polynucleotide that has homology with the target region of sgRNA_{target}.

Those of skill in the art will appreciate that the systems described herein can target genomic and extra-genomic (e.g., plasmid) sites.

In alternative embodiments, if is desirable to promote NHEJ, regulatory elements to conditionally express the Cas protein are associated with regulatory elements from genes actively expressed, for example, in G₀ or G₁. Examples of regulatory elements that can facilitate such expression of the Class 2 CRISPR-Cas systems of the present invention include regulatory sequences associated with a protein that drives the NHEJ pathway. Such proteins include, but are not limited to, Ku70, Ku80, DNA-PKcs, DNA Ligase IV, XRCC4, XLF, Artemis, DNA polymerase mu, DNA polymerase lambda, PNKP, Aprataxin, and APLF.

In further alternative embodiments, if is desirable to promote MMEJ, regulatory elements to conditionally express the Cas protein are associated with regulatory elements derived from genes whose expression is associated with MMEJ. Examples of such regulatory elements that facilitate expression of components of the MMEJ pathway include, but are not limited to, regulatory elements associated with expression of the following proteins: CtIP, PARP1, Pol θ, Lig1, and Lig3.

The present system also can include a locus-specific guide polynucleotide encoding a locus-specific guide RNA that can target the Cas protein (e.g., Cas9 protein or Cpf1 protein) to the desired locus in DNA. For Class 2 Type II CRISPR-Cas systems, a single-guide RNA or a dual-guide RNA is typically used. In preferred embodiments, the guide RNA is an sgRNA (e.g., as shown in FIG. 1C). For Class 2 Type V CRISPR-Cas systems, the guide RNA is typically a guide crRNA (e.g., as shown in FIG. 1D). In particular embodiments, the locus-specific guide polynucleotide can be operably linked to the first regulatory element such that the first regulatory element drives expression of a single transcript including a sequence encoding the Cas protein (e.g., Cas9 protein or Cpf1 protein), a transcript separator sequence, and a sequence encoding the locus-specific guide RNA.

Alternatively, the locus-specific guide polynucleotide can be operably linked to an additional copy of the first regulatory element or to a different regulatory element that is active in response to the first specific cell state. In this case, the system separately expresses a transcript encoding the locus-specific guide RNA. For Class 2 Type II CRISPR-Cas systems, when a dual-guide RNA is used (e.g., as shown in FIG. 1A) one or more polynucleotides can encode the crRNA and tracrRNA components.

In embodiments using a dual guide RNA, when a single polynucleotide encodes the crRNA and the tracrRNA a transcript separator is placed between the coding sequences for the crRNA and the tracrRNA. In other embodiments, the coding sequences for the crRNA and tracrRNA are each placed under the control of a promoter.

In preferred embodiments both the Cas9 protein and the sgRNA_{target} (or the dual guide crRNA_{Target}/tracrRNA) are controlled by expression using regulatory elements active in, for example, the same cell cycle. Typically, any arrangement that facilitates expression of the Cas protein (e.g., Cas9 protein or Cpf1 protein) and the guide RNA more or less at the same time is preferred.

In some embodiments, promoters regulated by molecules administered to the cells (exogenous induction) can also be used.

In yet other embodiments, the system does not include a locus-specific guide polynucleotide, but has, in its place, a multiple cloning site (MCS) so that the user can readily insert a guide polynucleotide sequence that is specific for the desired target locus. After insertion, the guide polynucleotide is operably linked to the first regulatory element.

To turn off transcription of a Cas protein (le., after sufficient Cas9 protein or Cpf1 protein has been produced to effect cleavage at the desired locus), the system can include a Cas-specific guide polynucleotide. The Cas-specific guide polynucleotide can encode, for example, a Cas9-specific guide RNA that can target a Cas9 to the Cas9 coding sequence and/or a Cpf1-specific guide RNA that can target a Cpf1 to the Cpf1 coding sequence. By targeting the Cas protein coding polynucleotide for cleavage, the Cas-specific guide RNA and associated Cas protein turn off transcription of the Cas gene. Generally, an sgRNA is most convenient for this purpose.

The expression of the Cas-specific guide (“sgRNA_{Cas}”) can be modulated by an operably linked second regulatory element (e.g., FIG. 2, 205, Promoter B) that is active in response to a second specific cell state. The first and second cell states can be the same or different. If the first and second cell states are the same, the first and second regulatory elements preferably differ in their responsiveness to the cell state. For example, in FIG. 2 the first regulatory element (FIG. 2, 203) drives expression of the Cas9 protein, and the second regulatory element (FIG. 2, 205) drives expression of a Cas9-specific guide RNA that targets the Cas9 protein to its own coding sequence. (In other embodiments, a Cpf1 protein and its cognate crRNA are used.) Because the second regulatory element acts as an off switch for expression of the Cas protein, the regulatory elements should generally be chosen so that the Cas-specific guide RNA expressed from the second promoter complexes with the Cas protein only after sufficient locus-specific Cas protein/guide ribonucleoprotein complexes have formed to facilitate, for example, site-specific HDR. This can be readily accomplished by using regulatory elements that have opposite responses to a cell state; if the first regulatory element is a promoter that is activated by the cell state, the second regulatory element can be a promoter that is repressed by the cell state. In this case, the presence of the cell state activates expression of the Cas protein, which is turned off when the cell state ceases to be present. In other embodiments, the expression from the promoter can be regulated by molecules introduced into the cells, for example, a molecule added to cell culture media.

FIG. 2 shows a first regulatory element (FIG. 2, 203) and operably linked first polynucleotide (encoding a Cas protein and a locus-specific guide RNA) and a second regulatory element (FIG. 2, 204) operably linked Cas-specific guide polynucleotide all in the same vector. This configuration is generally most convenient, but those of skill in the art appreciate that the first regulatory element and operably linked first polynucleotide and the second regulatory element and operably linked Cas-specific guide polynucleotide can be in different vectors.

In some embodiments, for example those intended for use in HDR, the engineered Class 2 CRISPR-Cas system includes a donor polynucleotide with homology to the target locus.

The following is another example of using the engineered Class 2 CRISPR-Cas systems described herein for enhancing directed DNA repair using a system in which the first regulatory element is operably linked to a Cas protein coding sequence (e.g., a Cas9 protein or a Cpf1 protein), a transcript separator, and a locus-specific, single-guide RNA as one transcript. In this case, the second regulatory element expresses multiple copies of the Cas-specific, sgRNA, separated from one another by a transcript separator sequence. Every round of transcription in the system produces multiple Cas-specific sgRNAs versus the one locus-specific sgRNA expressed from the first regulatory element. This difference ensures that, once the second regulatory element becomes active, the concentration of Cas-specific sgRNAs rapidly exceeds the concentration of the locus-specific sgRNAs. This concentration difference favors formation of Cas-specific nuclease complexes (e.g., Cas9/sgRNA_{Cas9} ribonucleoprotein complexes or Cpf1/crRNA_{Cpf1} ribonucleoprotein complexes) over formation of a locus-specific nuclease complex (e.g., Cas9/sgRNA_{target} ribonucleoprotein complexes or Cpf1/crRNA_{target} ribonucleoprotein complexes)), which provides a rapid and robust termination of expression of the Cas protein. This is exemplified in FIG. 3 using an engineered Class 2 Type II CRISPR-Cas9 system.

FIG. 3 illustrates use of the cas9 gene (FIG. 3, 300) and an sgRNA (FIG. 3, 302; “sgRNA_{target}”) capable of targeting a genomic region, as well as a transcript separator sequence (FIG. 3, 301) that liberates the cas9 RNA from the sgRNA_{target}. The Cas9-transcript separator-sgRNA_{target} transcript is operably linked to Promoter A (FIG. 3, 303; the direction of transcription is indicated by an arrow). Alternatively, the sgRNA_{target} can be expressed as a different transcript that is typically also operably linked to another copy of Promoter A. The system can also contain a polynucleotide encoding multiple sgRNAs (FIG. 3, 304, “sgRNA_{Cas9}”) that target the cas9 gene. This transcript is operably linked to Promoter B (FIG. 3, 305; the direction of transcription is indicated by an arrow). The vector backbone is shown as a solid black curve. This figure illustrates essentially the same system as is shown in FIG. 2, except in FIG. 3 the transcript operably linked to Promoter B is made up of multiple sgRNA_{Cas9} transcripts in order to affect stoichiometry of sgRNAs_{Cas9}. More sgRNA_{Cas9} relative to sgRNA_{target} means that more sgRNA_{Cas9} is able to bind to the Cas9 protein versus sgRNA_{target}. The sgRNA_{Cas9}/Cas9 protein complexes cleave the Cas9 protein coding sequence to terminate expression of Cas9 protein. The vector backbone is shown as a solid black curve.

Because NHEJ competes with HDR, in some embodiments of the invention, it is advantageous to produce the Cas protein (e.g., Cas9 protein or Cpf1 protein) under conditions where the HDR pathway is more competitive with the NHEJ pathway. One approach is to take advantage of natural fluctuations in the activity of the NHEJ and HDR pathways and time expression of Cas protein to occur when HDR is most active in the cell cycle. Mao, Z., et al., Cell Cycle 7(18):2902-2906 (2008) showed that NHEJ is active throughout the cell cycle, and NHEJ activity increases as cells progress from G₁ to G₂/M (G₁<S<G₂/M). HDR is nearly absent in G₁, most active in the S phase, and declines in G₂/M. Thus, expression of Cas protein is advantageous to promote HDR in S phase and G₂ and can be facilitated by use of regulatory elements associated with genes active during these cell cycle phases. Because natural fluctuations in the activity of the NHEJ may not be sufficient in a particular host cell, the embodiments of the present invention provide Class 2 CRISPR-Cas systems to facilitate transient repression of the NHEJ pathway (see, e.g., FIG. 5) and/or transient repression of the MMEJ pathway.

Some embodiments of the present invention take advantage of natural fluctuations in the activity of the NHEJ and HDR pathways. For example, a first regulatory element that controls expression of a Cas protein can include a repressor binding site. In this case, a second regulatory element can be operably linked to a repressor polynucleotide encoding a protein repressor of the first regulatory element. The second regulatory element can be a NHEJ pathway-specific regulatory element that drives expression of the protein that drives the NHEJ pathway. For example, the NHEJ pathway-specific regulatory element can be a promoter or enhancer from the Ku gene, so that a transcription factor that activates Ku expression also activates expression of the repressor protein; thus, when Ku is expressed, expression of the Cas protein is repressed. When transcriptional expression of Ku protein is reduced, thus reducing production of the repressor, expression of the Cas protein is facilitated. Expressing the Cas protein and a locus-specific guide RNA leads to cleavage of the target locus. Regulatory elements associated with the expression of proteins associated with the MMEJ pathway can be similarly used.

FIG. 4 illustrates a system of this type for enhancing HDR. This example uses an engineered Class 2 Type II CRISPR-Cas9 system. In this embodiment, the Ku protein is required for NHEJ. In the example shown in FIG. 4, Promoter B, here a Ku-specific promoter (FIG. 4, 406; the direction of transcription is indicated by an arrow), is placed upstream of the lacI gene (FIG. 4, 405). The lacI gene codes for the lac repressor protein (lad protein). Thus, when Ku protein is being expressed, so is lad protein. The lac repressor protein (FIG. 4, 407) binds to the lacO operator sequence (FIG. 4, 404), which is located between Promoter A (FIG. 4, 403; the direction of transcription is indicated by an arrow) and the cas9 gene (FIG. 4, 400). The system also contains an sgRNA (FIG. 4, 402; “sgRNA_{target}”) capable of targeting a genomic region, as well as a transcript separator sequence (FIG. 4, 401) that liberates the cas9 RNA from the sgRNA_{target}. The Cas9-transcript separator-sgRNA_{target} transcript is operably linked to Promoter A. When the lac repressor protein is bound to the lacO operator sequence, Cas9 is not expressed because the lac repressor protein binding prevents RNA polymerase from binding the transcription start site. As the cell progresses through the cell cycle, the transcription factor (FIG. 4, 408) that activates expression from the Ku-specific promoter ceases to be expressed (or is expressed at lower levels). This results in a reduction in lacI gene expression and a reduction in expression of the Ku gene (FIG. 4, 409) located in genomic DNA (FIG. 4, 410) of the cell. Ultimately, this allows for Cas9 protein and the sgRNA_{target} to be expressed. When Ku gene expression is turned off or turned down, the NHEJ pathway is less active and HDR occurs with greater efficiency. The vector backbone is shown as a solid black curve.

Expressing the Cas9 protein and the locus-specific Cas9 leads to cleavage of the target locus. Down-regulation of the NHEJ pathway can increase the likelihood that repair of the break will be by HDR, rather than NHEJ. As with the previously discussed systems, these regulatory elements and their operably linked sequences can be in the same vector, as shown in FIG. 4, or in different vectors.

In some embodiments, a Cpf1 protein and its cognate crRNA are used. In some embodiments, the system has a multiple cloning site in place of the locus-specific guide polynucleotide to permit users to insert their own locus-specific guide polynucleotide.

FIG. 4 exemplifies a Cas9 protein and a locus-specific guide RNA expressed from the first regulatory element as a single transcript. Alternatively, the locus-specific guide polynucleotide can be operably linked to an additional copy of the first regulatory element or to a different regulatory element for expression as a separate transcript. If a different regulatory element is used, it generally responds to the level of the protein that drives the NHEJ pathway in a similar manner to the first regulatory element.

In some embodiments, HDR is enhanced by actively transiently repressing DNA repair pathway components that, when repressed transiently, facilitate higher levels of HDR relative to when the components are expressed at wild-type levels in a host cell. NHEJ and MMEJ are examples of such repair pathways. Methods for identifying further such DNA repair pathway components are described in Example 3. For example, the DNA repair pathway (e.g., the NHEJ pathway) is transiently repressed so that DNA repair cannot efficiently occur through that pathway when a Cas protein (e.g., Cas9 protein or Cpf1 protein) is expressed. One way of achieving this is to use a binding-competent, but catalytically inactive, Cas protein that targets a gene that encodes a protein that drives the DNA repair pathway (e.g., NHEJ), thereby inhibiting transcription of that gene and down-regulating the pathway. In particular embodiments, a catalytically inactive variant of Cas9 dCas9) or a catalytically inactive variant of Cpf1 dCpf1) can be used as a targeted repressor. In further embodiments, a catalytically inactive Cas protein coding sequence can be fused to a coding sequence for a repressor moiety that is particular to a gene that encodes a protein that drives the DNA repair pathway (e.g., KRAB repressor moiety coding sequences).

Systems of this type can include, in addition to a first regulatory element driving expression of Cas protein, a second regulatory element driving expression of an inactive Cas protein. In some embodiments, these two regulatory elements are active in response to the same cell state. These expression cassettes can be in the same or different vectors. Each Cas protein (e.g., the active Cas9 and the inactive Cas9, or the active Cpf1 and the inactive Cpf1) should selectively associate with its own cognate guide RNA. For example, to ensure that each selectively associates with the proper guide RNA, a Cas9 protein and a dCas9 protein can be derived from different species, a Cas9 protein with its cognate guide RNA and a dCpf1 protein and its cognate guide RNA can be used, a Cpf1 protein with its cognate guide RNA and a dCas9 protein and its cognate guide RNA can be used, or a Cpf1 protein and dCpf1 protein can be derived from different species.

FIG. 5 illustrates an engineered Class 2 CRISPR-Cas system for enhancing HDR where Cas protein expression is coupled to repression of the NHEJ pathway. This example uses an engineered Class 2 Type II CRISPR-Cas9 system, comprising two Cas9 protein coding sequences. The system comprises the cas9 gene (FIG. 5, 500), which is translated into the Cas9 protein (FIG. 5, 507). The system contains an sgRNA (FIG. 5, 502; “sgRNA_{target}”) capable of targeting a genomic region (i.e., a target DNA sequence (FIG. 5, 508) in the genomic DNA of a cell (FIG. 5, 509)), as well as a transcript separator sequence (FIG. 5, 501) that liberates the cas9 RNA from the sgRNA_{target}. The Cas9-transcript separator-sgRNA_{target} polynucleotide is operably linked to Promoter B (FIG. 5, 503; the direction of transcription is indicated by an arrow). The system further comprises a dcas9 gene (FIG. 5, 504), which is a polynucleotide encoding an RNA that is translated into a dCas9 protein (FIG. 5, 510). dCas9 protein is a catalytically inactive Cas9 protein that is capable of binding to a target nucleic acid sequence but does not cleave the target nucleic acid sequence. The system contains an sgRNA (FIG. 5, 505; “sgRNA_{Ku}”) capable of targeting the Ku gene (FIG. 5, 512; i.e., a target DNA sequence (FIG. 5, 513) in the genomic DNA of the cell (FIG. 5, 509)), as well as a transcript separator sequence (FIG. 5, 501) that liberates the cas9 RNA from the sgRNA_{target}. The dCas9-transcript separator-sgRNA_{Ku} polynucleotide is operably linked to Promoter A (FIG. 5, 506; the direction of transcription is indicated by an arrow). dCas9 protein associates with sgRNA_{Ku} to form a ribonucleoprotein complex that binds the Ku gene and blocks Ku gene transcription.

Examples of binding sites within the Ku gene to block transcription include, but are not limited to, a transcription start site, and/or a promoter region. The active Cas9 associates with sgRNA_{target} to form a ribonucleoprotein complex that cleaves the target DNA sequence. In some embodiments, orthogonal Cas9 protein/sgRNA_{target} and dCas9 protein/sgRNA_{Ku} backbone pairings are used to avoid cross-talk between the two ribonucleoprotein complexes (e.g., by using Cas9 coding sequences from different species, or by using a dCas9 protein coding sequence and a Cpf1 coding sequence, or by using a dCpf1 coding sequence and a Cas9 protein coding sequence). As shown in FIG. 5, sgRNA_{Ku} guides dCas9 to the Ku gene, wherein binding of the dCas9/sgRNA_{Ku} complex inhibits Ku transcription. Alternatively or additionally, a dCas9 can be fused to a transcriptional repressor domain, such as KRAB, in which case an sgRNA_{Ku} is designed to target the dCas-KRAB fusion protein/sgRNA_{Ku} complex to a Ku enhancer domain where the complex represses Ku transcription. The vector backbone in FIG. 5 is shown as solid black curve.

Further embodiments include Promoter A and Promoter B each comprising a regulatory element wherein the regulatory element can be the same or different and is active in response to a first cell cycle phase of the host cell (e.g., a regulatory element active in S and/or G₂ when expression of proteins that are part of the HDR pathway are most expressed). In such embodiments, when the Cas9 protein and sgRNA_{target} are expressed, the expression of Ku protein is repressed in the same cell cycle phase by the dCas9 protein/sgRNA_{Ku} complex, thus suppressing NHEJ pathway and increasing efficiency of HDR.

In yet a further embodiment, this system is well suited to exogenous induction (e.g., using a molecule introduced into cell culture media) of Promoter A and/or Promoter B rather than tying expression to a natural cell state. For example, expression from Promoter A can be activated first and expression from Promoter B can be activated after expression of the Ku protein is suppressed.

As described in FIG. 5, a first regulatory element is operably linked to a polynucleotide encoding, in order, the active Cas9-a transcript separator-the sgRNA_{target}. In the figure, Cas9 and sgRNA_{target} are expressed from the first regulatory element as a single transcript. Alternatively, the sgRNA_{target} can be operably linked to an additional copy of the first regulatory element or to a different regulatory element for expression as a separate transcript. If a different regulatory element is used, it will generally be active in response to the same specific cell state as the first regulatory element so that expression of both components of the system occurs at more or less the same time.

Similarly, the second regulatory element in FIG. 5 is operably linked to a polynucleotide encoding, in order, an inactive Cas9 (dCas9)-a transcript separator-a guide polynucleotide encoding a guide RNA, wherein the guide RNA can target a gene encoding a protein that drives the NHEJ pathway. The NHEJ pathway-specific guide polynucleotide can be expressed with the inactive Cas9 in a single transcript, as shown in FIG. 5, or as a separate transcript, provided that expression of both components of the system occurs at more or less the same time.

In a transformation experiment, DNA is introduced into a small percentage of target cells only. Genes that encode selectable markers are useful and efficient in identifying cells that are stably transformed when the cells receive and integrate a transgenic DNA construct into their genomes. Preferred marker genes provide selective markers that confer resistance to a selective agent, such as an antibiotic or herbicide. Illustrative selective markers can confer antibiotic resistance (e.g., G418 bleomycin, kanamycin, hygromycin), biocide resistance, or herbicide resistance (e.g., glyphosate). Examples include, but are not limited to, a neo gene, which confers kanamycin resistance and can be selected for using kanamycin or G418; a bar gene, which confers bialaphos resistance; a mutant EPSP synthase gene, which confers glyphosate resistance; a nitrilase gene, which confers resistance to bromoxynil; a mutant acetolactate synthase gene (ALS), which confers imidazolinone or sulphonylurea resistance; and DHFR gene, which confers methotrexate-resistance.

A screenable marker, which may be used to monitor expression, may also be included in a vector. Screenable markers include, but are not limited to, a β-glucuronidase or uidA gene (GUS), which encodes an enzyme for which various chromogenic substrates are known; an R-locus gene, which encodes a product that regulates the production of anthocyanin pigments (red color) in plant tissues; a β-lactamase gene, which encodes an enzyme for which various chromogenic substrates are known (e.g., PADAC, a chromogenic cephalosporin); a luciferase gene; an xylE gene, which encodes a catechol dioxygenase that converts chromogenic catechols; an α-amylase gene; a tyrosinase gene, which encodes an enzyme that oxidizes tyrosine to DOPA and dopaquinone, which in turn condenses to melanin; and an α-galactosidase gene, which encodes an enzyme that catalyzes a chromogenic α-galactose substrate.

Expression vectors for host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, and viral vectors (including retroviral, lentivirus, adenoviral, adeno-associated and herpes simplex virus vectors) for cell transformation and gene expression and methods to easily allow cloning of such polynucleotides. Illustrative plant transformation vectors include those derived from a Ti plasmid of Agrobacterium tumefaciens (Lee, L. Y., al., Plant Physiol. 146(2): 325-332 (2008)). Also useful and known in the art are Agrobacterium rhizogenes plasmids. For example, SNAPGENE™ (GSL Biotech LLC, Chicago, Ill.; snapgene.com/resources/plasmid_files/your_time_is_valuable/) provides an extensive list of vectors, individual vector sequences, and vector maps, as well as commercial sources for many of the vectors.

Viral vectors are particularly convenient for use in the pharmaceutical compositions of the disclosure. Exemplary viruses for this purpose can include lentivirus, retrovirus, adenovirus, herpes simplex virus I or II, parvovirus, reticuloendotheliosis virus, and adeno-associated virus (AAV).

To facilitate viral delivery, any of the systems described herein can be packaged into a viral particle using conventional methods. Packaging cells are typically used to form virus particles that are capable of infecting a host cell. Exemplary cells include 293 cells, which package adenovirus, and .psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, i.e., rep and cap, but lacking ITR sequences. The cell line may also be infected with adenovirus as a helper virus. The helper virus promotes replication of the AAV vector and expression of AAV genes from the helper plasmid. The helper plasmid is not packaged in significant amounts due to a lack of ITR sequences. Contamination with adenovirus can be reduced by, for example, heat treatment to which adenovirus is more sensitive than AAV. Additional methods for the delivery of nucleic acids to cells are known to those skilled in the art. See, for example, U.S. Published Patent Application No. 2003-0087817, published 8 May 2003.

Lentivirus is a member of the Retroviridae family and is a single-stranded RNA virus, which can infect both dividing and nondividing cells as well as provide stable expression through integration into the genome. To increase the safety of lentivirus, components necessary to produce a viral vector are split across multiple plasmids. Transfer vectors are typically replication incompetent and may additionally contain a deletion in the 3′LTR, which renders the virus self-inactivating after integration. Packaging and envelope plasmids are typically used in combination with a transfer vector. For example, a packaging plasmid can encode combinations of the Gag, Pol, Rev, and Tat genes. A transfer plasmid can comprise viral LTRs and the psi packaging signal. The envelope plasmid comprises an envelope protein (usually vesicular stomatitis virus glycoprotein, VSV-GP, because of its wide infectivity range).

Lentiviral vectors based on human immunodeficiency virus type-1 (HIV-1) have additional accessory proteins that facilitate integration in the absence of cell division. HIV-1 vectors have been designed to address a number of safety concerns. These include separate expression of the viral genes in trans to prevent recombination events leading to the generation of replication-competent viruses. Furthermore, the development of self-inactivating vectors reduces the potential for transactivation of neighboring genes and allows the incorporation of regulatory elements to target gene expression to particular cell types (see, e.g., Cooray, S., et al., Methods Enzymol. 507:29-57 (2012)).

A number of vectors for use in mammalian cells are commercially available, for example: pcDNA3 (Life Technologies, South San Francisco, Calif.); customizable expression vectors, transient vectors, stable vectors, and lentiviral vectors (DNA 2.0, Menlo Park, Calif.); and pFN10A (ACT) FLEXI® (Promega, Madison, Wis.) vector. Furthermore, the following elements can be incorporated into vectors for use in mammalian cells: RNA polymerase II promoters operatively linked to Cas9 coding sequences; RNA polymerase III promoters operably linked to coding sequences for guide RNAs; and selectable markers (e.g., G418, gentamicin, kanamycin and ZEOCIN™ (Life Technologies, Grand Island, N.Y.)). Nuclear targeting sequences can also be added, for example, to Cas9 protein coding sequences.

Regulatory elements, as discussed herein, can direct expression in a temporal-dependent manner (e.g., in a cell-cycle dependent or developmental stage-dependent manner). In some embodiments, vectors comprise regulatory elements associated with one or more RNA polymerase III promoter, one or more RNA polymerase II, one or more RNA polymerase I promoters, or combinations thereof. Examples of mammalian RNA polymerase III promoters include, but are not limited to, the following: U6 and H1 promoters. Examples of RNA polymerase II promoters and RNA polymerase I promoters are well known in the art.

Example 1 describes a method for designing vectors that provide conditional expression, in response to specific a cellular state, of a Cas protein and guide RNA species.

Numerous mammalian cell lines have been utilized for expression of gene products including HEK 293 (Human embryonic kidney) and CHO (Chinese Hamster Ovary). These cell lines can be transfected by standard methods (e.g., using calcium phosphate or polyethyleneimine (PEI), or electroporation). Other typical mammalian cell lines include, but are not limited to, the following cell lines: HeLa, U2OS, 549, HT1080, CAD, P19, NIH 3T3, L929, N2a, Human embryonic kidney 293 cells, MCF-7, Y79, SO-Rb50, Hep G2, DUKX-X11, J558L, and Baby Hamster Kidney (BHK) cells.

Any of the systems described herein can be introduced into a host cell of any type or an organism.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery. For ease of discussion, “transfection” is used below to refer to any method of introducing polynucleotides into a host cell.

Preferred methods for introducing polynucleotides plant cells include microprojectile bombardment and Agrobacterium-mediated transformation. Alternatively, other non-Agrobacterium species (e.g., Rhizobium) and other prokaryotic cells that are able to infect plant cells and introduce heterologous polynucleotides into the genome of the infected plant cell can be used. Other methods include electroporation, liposome-mediated transfection, transformation using pollen or viruses, and chemicals that increase free DNA uptake, or free DNA delivery using microprojectile bombardment. See, e.g., Narusaka, Y., et al, Chapter 9, in Transgenic Plants—Advances and Limitations, edited by Yelda, O., ISBN 978-953-51-0181-9 (2012).

In some embodiments, a host cell is transiently or non-transiently transfected with one or more systems described herein. In some embodiments, a cell is transfected as it naturally occurs in a subject. In some embodiments, a cell that is transfected is taken from a subject, e.g., a primary cell. In some embodiments, the primary cell is cultured and/or is returned after ex vivo transfection to the same subject (autologous treatment) or to a different subject.

Example 2 describes a method for introducing a Cas protein expressing vector as well as a donor polynucleotide into mammalian cells. The example also describes a method for validating the incorporation of the donor polynucleotide into the host cell.

In some embodiments, a cell transfected with one or more systems described herein is used to establish a new cell or cell line including one or more vector-derived sequences. In some embodiments, a cell transiently transfected with one or more systems described herein and modified through the activity of the system, is used to establish a new cell or cell line including cells containing a genomic modification but lacking any other exogenous sequence. In certain embodiments, a transfected host cell is cultured under conditions suitable for the transfected system to incorporate a donor polynucleotide into DNA in this host cell. At least one aspect of the culture conditions permits, promotes, or supports a specific cell state that is not continuously present in the host cell. In some embodiments, the culture cell conditions permit or promote a specific cellular state that activates the first regulatory element to express a Cas protein (e.g., a Cas9 protein or a Cpf1 protein). In some embodiments, an exogenous stimulus is introduced into the culture, and this activates expression of a Cas protein. In other embodiments, an exogenous stimulus is introduced into a culture to facilitate removal of active Cas protein in a cell cycle specific manner. Example 5 describes the combined use of a cell cycle regulated promoter and Cas protein depletion using a chemically controlled tag.

The Cas protein cleaves host cell DNA (genomic or other) at the selected target locus, and the donor polynucleotide is incorporated into the host cell DNA, preferably by HDR, which can be result insertions, deletions, or mutations of bases in the host cell DNA. This approach can be used, for example, for gene correction, gene replacement, gene tagging, transgene insertion, gene disruption, gene mutation, mutation of gene regulatory sequences, and so on. In some embodiments, incorporation of the donor polynucleotide into the host cell occurs with an efficiency greater than achieved by constitutive expression of a Cas protein in the presence of the donor polynucleotide in the host cell. In various embodiments, the efficiency of the donor polynucleotide incorporation is improved by 3, 5, 8, 10, 13, 15, 18, 20, 23, 25, 28, 30, 33, 35, 38, 40, 43, 45, 48, 50, 53, 55, 58, 60, 63, 65, 68, 70, 73, 75, 78, 80, 83, 85, 88, 90, 93, 95, 98, or 100% relative to not using a regulatory element that is active in response to a specific cell state. In some embodiments, the percentage improvement falls within a range bounded by any of these values.

In some embodiments, the transfected host cell is cultured to produce a progeny cell that includes the incorporated donor polynucleotide (or portion or copy thereof). In some embodiments, culturing produces a population of cells, where each cell includes the incorporated donor polynucleotide (or a portion thereof). Examples of such cells include myeloid cells (e.g., monocytes, macrophages, neutrophils, basophils, eosinophils, erythrocytes, dendritic cells, and megakaryocytes or platelets) and lymphoid cells (e.g., T cells, B cells, and natural killer cells). Examples of progenitor cells include multipotent, oligopotent, and unipotent hematopoietic progenitor cells, adipose tissue stem cells, and umbilical cord blood stem cells.

Example 4 describes creation of a stable cell line containing an expression cassette integrated at a genomic location. A selected gene is transiently repressed (e.g., a gene discovered in the screen described in Example 3) to facilitate integration of a large cassette at high efficiency in a predefined locus of a T cell.

Any of the components of the systems described above can be incorporated into a kit, optionally including one or more reagents useful in conjunction with the system to carry out DNA repair. In some embodiments, a kit includes a package with one or more containers holding the kit elements, as one or more separate compositions or, optionally, as admixture where the compatibility of the components will allow. In some embodiments, kits also comprise a buffer and/or preservatives. Illustrative kits comprise Class 2 CRISPR-Cas polynucleotides of the present invention comprising regulatory elements and coding sequences for a Cas protein (e.g., a Cas9 or a Cpf1 protein) and/or or a polynucleotide encoding a guide, vector or vectors comprising the Class 2 CRISPR-Cas polynucleotides of the present invention, and optionally a donor polynucleotide or a set of different donor polynucleotides.

Furthermore, kits can further comprise instructions for using the systems described herein, e.g., to carry out DNA repair. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), RF tags, and the like. Instructions can also include the address of an internet site that provides the instructions.

A system or cell, as described herein can be used as a pharmaceutical composition, where it is, in some embodiments, formulated with a pharmaceutically acceptable excipient. As used with reference to a pharmaceutical composition, “active agent” refers to a Class 2 CRISPR-Cas system (e.g., a Class 2 Type II CRISPR-Cas system or a Class 2 Type V CRISPR-Cas system) or cells modified by use of this system.

Illustrative excipients include carriers, stabilizers, diluents, dispersing agents, suspending agents, thickening agents, and the like. The pharmaceutical composition can facilitate administration of the active agent to an organism. Pharmaceutical compositions can be administered in therapeutically effective amounts by various forms and routes including, for example, intravenous, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the active agent directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

Therapeutically effective amounts of the active agents described herein can be administered in pharmaceutical compositions to a subject having a disease or condition to be treated. A therapeutically effective amount can vary widely depending on the severity of the disease, the age and relative health of the subject, the potency of the active agents used, and other factors. The active agents can be used singly or in combination with one or more therapeutic agents as components of mixtures.

Pharmaceutical compositions can be formulated using one or more pharmaceutically acceptable excipients, which facilitate processing of the active agent into preparations that can be used pharmaceutically. Formulation can be modified depending upon the route of administration chosen.

Pharmaceutical compositions containing active agents described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the disease or condition. Amounts effective for this use can vary based on the severity and course of the disease or condition, previous therapy, the health status, weight, and response to the drugs of the subject, and the judgment of the treating physician.

In some embodiments, an active agent, such as a vector, can be packaged into a biological compartment for administration to a subject. A biological compartment including the active agent can be administered to a subject. Biological compartments can include, but are not limited to, nanospheres, liposomes, quantum dots, nanoparticles, microparticles, nanocapsules, vesicles, polyethylene glycol particles, hydrogels, and micelles.

The systems described herein can be used to generate non-human transgenic organisms by site-specifically introducing a selected polynucleotide sequence at a DNA target locus in the genome to generate a modification of the genomic DNA. The transgenic organism can be an animal or a plant.

A transgenic animal is typically generated by introducing the system into a zygote cell. A basic technique, described with reference to making transgenic mice (Cho, A., et al, “Generation of Transgenic Mice,” Current Protocols in Cell Biology, CHAPTER.Unit-19.11 (2009)), involves five basic steps: first, preparation of a system, as described herein, including a suitable donor polynucleotide; second, harvesting of donor zygotes; third, microinjection of the system into the mouse zygote; fourth, implantation of microinjected zygotes into pseudo-pregnant recipient mice; and fifth, performing genotyping and analysis of the modification of the genomic DNA established in founder mice. The founder mice will pass the genetic modification to any progeny. The founder mice are typically heterozygous for the transgene. Mating between these mice will produce mice that are homozygous for the transgene 25% of the time.

Methods for generating transgenic plants are also well known. A transgenic plant generated, e.g., using Agrobacterium transformation methods typically contains one transgene inserted into one chromosome. It is possible to produce a transgenic plant that is homozygous with respect to a transgene by sexually mating (i.e., selfing) an independent segregant transgenic plant containing a single transgene to itself, for example an F0 plant, to produce F1 seed. Plants formed by germinating F1 seeds can be tested for homozygosity. Typical zygosity assays include, but are not limited to, single nucleotide polymorphism assays and thermal amplification assays that distinguish between homozygotes and heterozygotes.

As an alternative to using a system described herein for the direct transformation of a plant, transgenic plants can be formed by crossing a first plant that has been transformed with a system with a second plant that has never been exposed to the system. For example, a first plant line containing a transgene can be crossed with a second plant line to introgress the transgene into the second plant line, thus forming a second transgenic plant line.

The Class 2 CRISPR-Cas systems described herein provide a tool for plant breeders. Accordingly, one skilled in the art can analyze the genome of sources of resistance genes and use the present invention in varieties having desired traits or characteristics to induce the rise of resistance genes; this result can be achieved with more precision than by using previous mutagenic agents, thereby accelerating and enhancing plant breeding programs.

Various embodiments contemplated herein include, but are not be limited to, one or more of the following.

### Embodiment 1

A Type II CRISPR-Cas9 system including one or more vectors for use in directing DNA repair at a specific locus in a host cell genome, the one or more vectors including: a first polynucleotide encoding Cas9, wherein the first polynucleotide is operably linked to a first regulatory element that is active in response to a first specific cell state of the host cell; and a locus-specific guide polynucleotide encoding a locus-specific guide RNA that is capable of forming a complex with Cas9.

### Embodiment 2

The system of embodiment 1, wherein the locus-specific guide RNA is capable of targeting the Cas9 to the specific locus in the host cell genome.

### Embodiment 3

The system of embodiment 1, wherein the locus-specific guide polynucleotide includes a multiple cloning site (MCS), wherein a sequence of the specific locus can be inserted to express a locus-specific guide RNA that is capable of targeting the Cas9 to the specific locus.

### Embodiment 4

The system of embodiment 3, wherein the MCS is located so that a polynucleotide cloned into the MCS is operably linked to the first regulatory element or operably linked to an additional copy of the first regulatory element or to a different regulatory element that is active in response to the first specific cell state.

### Embodiment 5

The system of any of embodiments 1-4, wherein the first specific cell state is transient in the host cell.

### Embodiment 6

The system of any of embodiments 1-5, wherein the locus-specific guide polynucleotide is operably linked to the first regulatory element, which drives expression of a single transcript including a sequence encoding the Cas9, a transcript separator sequence, and a sequence encoding the locus-specific guide RNA.

### Embodiment 7

The system of any of embodiments 1-5, wherein the locus-specific guide polynucleotide is operably linked to an additional copy of the first regulatory element or to a different regulatory element that is active in response to the first specific cell state, wherein the system expresses a transcript encoding the Cas9 separately from a transcript encoding the locus-specific guide RNA.

### Embodiment 8

The system of any of embodiments 1-7, additionally including: a Cas9-specific guide polynucleotide encoding a Cas9-specific guide RNA that can target the Cas9 to the first polynucleotide, wherein the Cas9-specific guide polynucleotide is operably linked to a second regulatory element that is active in response to a second specific cell state of the host cell.

### Embodiment 9

The system of embodiment 8, wherein the first and second cell states are different.

### Embodiment 10

The system of any of embodiments 1-9, wherein each regulatory element includes one or more of a regulatory element selected from the group consisting of a promoter, an enhancer, or a repressor binding sequence.

### Embodiment 11

The system of any of embodiments 8-10, wherein the first regulatory element and operably linked first polynucleotide and the second regulatory element and operably linked Cas9-specific guide polynucleotide are in the same vector.

### Embodiment 12

The system of any of embodiments 8-10, wherein the first regulatory element and operably linked first polynucleotide and the second regulatory element and operably linked Cas9-specific guide polynucleotide are in different vectors.

### Embodiment 13

The system of any of embodiments 8-12, wherein the Cas9-specific guide polynucleotide encodes multiple copies of the Cas9-specific guide RNA, wherein sequences encoding the copies are separated by a transcript separator sequence.

### Embodiment 14

The system of any of embodiments 1-7, wherein the first cell state includes the level of a protein that drives the NHEJ pathway in the host cell.

### Embodiment 15

The system of embodiment 14, wherein the protein that drives the NHEJ pathway includes a Ku protein.

### Embodiment 16

The system of either of embodiments 14 or 15, additionally including a repressor polynucleotide encoding a protein repressor of the first regulatory element, wherein the repressor polynucleotide is operably linked to a NHEJ pathway-specific regulatory element that drives expression of the protein that drives the NHEJ pathway.

### Embodiment 17

The system of embodiment 16, wherein the first regulatory element and operably linked first polynucleotide and the NHEJ pathway-specific regulatory element and operably linked repressor polynucleotide are present on the same vector.

### Embodiment 18

The system of embodiment 16, wherein the first regulatory element and operably linked first polynucleotide and the NHEJ-specific regulatory element and operably linked repressor polynucleotide are present on different vectors.

### Embodiment 19

The system of any of embodiments 16-18, wherein the first regulatory element includes a lacO operator sequence, and the repressor polynucleotide includes a lacI gene sequence, wherein the NHEJ pathway-specific regulatory element, when active, drives expression of a lac repressor, which binds to the lacO operator sequence and represses transcription of the Cas9.

### Embodiment 20

The system of any of embodiments 1-7, wherein the system additionally includes a second polynucleotide encoding an inactive Cas9, wherein the second polynucleotide is operably linked to a second regulatory element.

### Embodiment 21

The system of embodiment 20, wherein the first regulatory element and operably linked first polynucleotide and the second regulatory element and operably linked second polynucleotide are in the same vector.

### Embodiment 22

The system of embodiment 20, wherein the first regulatory element and operably linked first polynucleotide and the second regulatory element and operably linked second polynucleotide are in different vectors.

### Embodiment 23

The system of any of embodiments 20-22, wherein the system additionally includes a NHEJ pathway-specific guide polynucleotide encoding a NHEJ pathway-specific guide RNA that can target a gene that encodes a protein that drives the NHEJ pathway.

### Embodiment 24

The system of embodiment 23, wherein the protein that drives the NHEJ pathway includes a Ku protein.

### Embodiment 25

The system of either of embodiments 23 or 24, wherein the NHEJ pathway-specific guide polynucleotide is operably linked to the second regulatory element, which drives expression of a single transcript including a sequence encoding the inactive Cas9, a transcript separator sequence, and a sequence encoding the NHEJ pathway-specific guide RNA.

### Embodiment 26

The system of either of embodiments 23 or 24, wherein the NHEJ pathway-specific guide polynucleotide is operably linked to an additional copy of the second regulatory element or to a different regulatory element, wherein the system expresses a transcript encoding the inactive Cas9 separately from a transcript encoding the NHEJ-specific guide RNA.

### Embodiment 27

The system of any of 23-26, wherein the Cas9 selectively binds the locus-specific guide RNA, and the inactive Cas9 selectively binds the NHEJ pathway-specific guide RNA.

### Embodiment 28

The system of any of embodiments 23-27, wherein the inactive Cas9 is fused to a repressor moiety, and the NHEJ pathway-specific guide RNA targets an enhancer domain of the gene.

### Embodiment 29

The system of any of embodiments 20-28, wherein all regulatory elements are active in response to the same cell state.

### Embodiment 30

The system of any preceding embodiment, wherein the cell state includes a particular phase of the cell cycle.

### Embodiment 31

The system of embodiment 30, wherein the particular phase of the cell cycle is S or G₂.

### Embodiment 32

The system of embodiment 30, wherein the particular phase of the cell cycle is G₁, G₀, or M.

### Embodiment 32

The system of any preceding embodiment, wherein the cell state results from an exogenous stimulus.

### Embodiment 33

The system of any preceding embodiment, additionally including a donor polynucleotide that is capable of being incorporated into the specific locus.

### Embodiment 34

The system of embodiment 33, wherein introduction of the system into the host cell results in incorporation of a sequence from the donor polynucleotide into the host cell genome.

### Embodiment 35

The system of any preceding embodiment, wherein the vector(s) comprise(s) one or more plasmids.

### Embodiment 36

The system of any preceding embodiment, wherein the vector(s) comprise(s) one or more viral vectors.

### Embodiment 37

A kit including the system of any of embodiments 1-36, wherein the kit additionally includes instructions for using the system to incorporate a sequence from a donor polynucleotide into a host cell genome.

### Embodiment 38

A host cell including the system of any of embodiments 1-36.

### Embodiment 39

The host cell of embodiment 38, wherein the host cell is ex vivo.

### Embodiment 40

The host cell of either of embodiments 38 or 39, wherein the host cell includes a eukaryotic cell.

### Embodiment 41

The host cell of embodiment 40, wherein the host cell includes an animal cell.

### Embodiment 42

The host cell of embodiment 41, wherein the host cell includes a stem cell or induced pluripotent cell.

### Embodiment 43

The host cell of either of embodiments 38, wherein the host cell includes a prokaryotic cell.

### Embodiment 44

The host cell of embodiment 43, wherein the prokaryotic cell includes a bacterial cell.

### Embodiment 45

The host cell of either of embodiments 38 or 39, wherein the host cell includes a plant cell.

### Embodiment 46

The host cell of any of embodiments 38-45, wherein the system has operated to incorporate a sequence from a donor polynucleotide into the specific locus in the host cell genome.

### Embodiment 47

A pharmaceutical composition including the system of any of embodiments 1-36 and a pharmaceutically acceptable excipient.

### Embodiment 48

A pharmaceutical composition including the host cell of any of embodiments 38-46 and a pharmaceutically acceptable excipient.

### Embodiment 49

A plant composition including a seed, wherein the seed includes the system of any of embodiments 1-36.

### Embodiment 50

A plant composition including a seed, wherein the seed includes the cell of any of embodiments 38-46.

### Embodiment 51

A method including introducing the system of any of any of embodiments 1-36 into a host cell.

### Embodiment 52

The method of embodiment 51, wherein the system is introduced into the host cell ex vivo.

### Embodiment 53

The method of either of embodiments 51 or 52, wherein the host cell includes an animal cell.

### Embodiment 54

The method of embodiment 53, wherein the host cell includes a stem cell or induced pluripotent cell.

### Embodiment 55

The method of either of embodiments 51 or 52, wherein the host cell includes a plant cell.

### Embodiment 56

The method of any of embodiments 51-55, wherein the method includes culturing the host cell.

### Embodiment 57

The method of any of embodiments 51-56, the method including introducing a donor polynucleotide into the host cell, wherein a sequence from the donor polynucleotide becomes incorporated into the specific locus in the host cell genome.

### Embodiment 58

The method of embodiment 57, wherein the sequence is incorporated via HDR.

### Embodiment 59

The method of embodiment 58, wherein the sequences is incorporated in the S or G₂ phases of the cell cycle.

### Embodiment 61

The method of embodiment 57, wherein the sequence is incorporated via NHEJ.

### Embodiment 62

The method of embodiment 61, wherein the sequence is incorporated in the G₁, G₀, or M phases of the cell cycle.

Such embodiments can also comprise a Type V CRISPR-Cpf1 system using a Cpf1 protein and a Cpf1-specific guide polynucleotide, or combinations of a Cas9 protein/a Cas9-specific guide polynucleotide and a Cpf1 protein/a Cpf1-specific guide polynucleotide.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are illustrated in the following Examples. Efforts have been made to ensure accuracy with respect to numbers used (e.g., amounts, concentrations, percent changes, and the like) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only and are not intended to limit the scope of what the inventors regard as various aspects of the present invention.

Materials and Methods

Oligonucleotide sequences are provided to commercial manufacturers for synthesis (Integrated DNA Technologies, Coralville, Iowa; or Eurofins, Luxembourg).

### Example 1

### Design of a Vector Conditionally Expressing Cas9 Protein

This example describes a method for designing a vector that provides conditional expression, in response to specific a cellular state, of Cas9 protein and guide RNA species. The purpose of the conditional expression system is to increase the efficiency of HDR or other DNA repair pathways for engineering specific changes through substitution, insertion or deletion of nucleic acids into the target sequence of interest. Regulatory elements are selected and operably link to Cas9 and guide RNA sequences on vector(s) chosen for transfection into host cells. In this example, the cellular state described is the G₁/S transition of the cell cycle, and the target of the guide RNA spacer sequence is the FUT8 gene. Target sites are first selected from genomic DNA and guide RNAs are designed to target those selected sequences. Measurements are carried out to determine the level of target cleavage that has taken place. Illustrative basic steps are presented below. Not all of the following steps are required for every screening, nor must the order of the steps be as presented, and the screening can be coupled to other experiments, or form part of a larger experiment.

A. Selection of a Target DNA Sequence and a Corresponding Spacer

(i) Select a DNA target region, e.g., the FUT8 gene.

(ii) Identify all PAM sequences (e.g., ‘NGG’) within the selected genomic DNA region. This is done using, for example, the UCSC Genome Browser. This step can also be accomplished with a computational script that has access to the FASTA file for the human hg38 genome build (e.g., http site: hgdownload.soe.ucsc.edu/goldenPath/big38/bigZips/).

(iii) Identify and select one or more 20-nucleotide target nucleic acid sequences that are 5′ adjacent to a PAM sequence. Selection criteria can include but are not limited to: homology to other regions in the genome; percent G-C content; melting temperature; occurrences of homopolymer within the target nucleic acid sequence; and other criteria known to one skilled in the art. The UCSC genomic coordinates of the chosen FUT8 gene target sequence are chr14:65, 411,238-65,411, 257. Including the PAM sequence on the 5′ end, the UCSC genomic coordinates are chr14:65,411,238-65,411,260. The sequence of the chosen target sequence and PAM are 5′-GTACATCTTCTGTGTGATCTTGG-3′ (SEQ ID NO:4).

(iv) Append the required backbone of the guide RNA sequence (e.g., a single-guide RNA) to the 3′ end of the identified spacer sequence, excluding the “TGG” PAM sequence (5′-GTACATCTTCTGTGTGATCT-3′, SEQ ID NO:5). Together the spacer and backbone sequences form a guide RNA sequence.

Using the methods described here, a guide RNA can be programmed to target any genomic sequence of interest by engineering the sequence of the spacer region.

B. Selection of Regulatory Elements

Transcription factors that are active during the G₁ and S phases of the cell cycle are identified by using existing information sources, including the scientific literature or public databases such as ENCODE (www.encode.org), in view of the guidance of the present specification. One such example of a transcription factor is E2F1 as described in Johnson, D., et al., Nature 365(6444):349-52 (1993). In order to express Cas9 in response to the expression of E2F1, a promoter is chosen that contains the E2F1 consensus binding sequence. The consensus binding sequence is 5′-TTTCCCGC-3′ (or variants thereof, see, e.g., Tao, Y., et al., Molecular and Cellular Biology 17(12):6994-7007 (1997)). Using an online tool such as the Transcription Regulatory Element Database (cb.utdallas.edu/cgi-bin/TRED/tred.cgi?process=home), promoters containing the E2F binding sequence are identified. Several promoters may be tested in the Cas9-expressing vector to determine which one achieves greatest specificity of Cas9 expression within the desired phase of the cell cycle. Methods for testing expression can include flow cytometry using antibodies targeting either Cas9 or an epitope tag carried by Cas9, immunofluorescence, western blots or other methods known in the art. In this example, the chosen transcription factor is expressed in response to a particular cell cycle phase, but using the methods described here, it would be possible to select other transcription factors that are specifically expressed in response to any cellular state.

C. Plasmid Construction

The vector for Cas9 and guide RNA expression uses a S. pyogenes Cas9 sequence codon-optimized for expression in human cells, tagged at the C-terminus and optionally at the N-terminus, with at least one nuclear localization sequence (NLS). The Cas9 sequence can also contain an epitope tag at the N-terminus. In this example, the NLS is derived from SV40. The tagged Cas9 sequence is cloned into a vector adjacent to the chosen promoter sequence containing the E2F transcription factor-binding sequence. The vector is designed to include an sgRNA backbone sequence downstream of a cloning site, adjacent to a small RNA promoter sequence such as U6. The 20-nucleotide spacer to target the FUT8 gene is inserted into the cloning site located between the U6 promoter and the guide RNA backbone sequences.

### Example 2

### Introduction of the Conditional Cas9 Expression Vector into a Host Cell

This example describes a method for introducing a Cas9-expressing vector as well as a donor polynucleotide into HeLa cells. HeLa cells are an immortalized cell line of human epithelial cells. This example also describes a method for validating the incorporation of the donor polynucleotide into the host cell.

Examples of suitable media and culture conditions are described below. Modifications of these components and conditions will be understood by one of ordinary skill in the art in view of the teachings of the present specification.

A. Cell Culture

HeLa (ATCC CCL-2) cells can obtained from American Type Culture Collection (Manassas, Va.) and cultured in Dulbecco's modified Eagle medium (DMEM, Life Technologies, South San Francisco, Calif.), supplemented with 10% FBS (Life Technologies, South San Francisco, Calif.), 1% penicillin-streptomycin (Sigma-Aldrich, St. Louis, Mo.), 2 mM glutamine (Life Technologies, South San Francisco, Calif.) and cultured at 37° C., 5% CO2.

B. Transfection of Cells

HeLa cells are transiently transfected with the Cas9-containing vector as well as a donor polynucleotide with sequence homology to the selected target nucleic acid sequence (here, within the FUT8 gene) using TRANSIT®-LT1 transfection reagent (Mirus, Madison, Wis.). A non-transfected control is included. 72 hours after transfection, cells are trypsinized (Life Technologies, South San Francisco, Calif.) and dissociated with 10 nM EDTA-PBS (Lonza, Basel, Switzerland).

C. gDNA Sequencing

PCR primers are designed to amplify the portion of the FUT8 gene that contains the target DNA sequence from genomic DNA. Using isolated gDNA, a first PCR is performed using HERCULASE II Fusion DNA Polymerase (Agilent, Santa Clara, Calif.) with primers comprising universal adapter sequences. A second PCR is performed using the amplicons of the first round as template at 1/20^{th} the volume of the second PCR reaction volume. The second PCR uses a second set of primers comprising: sequences complementary to the universal adapter sequence of the first primer pair, a barcode index sequence unique to each sample, and a flow cell adapter sequence. PCR reactions are pooled to ensure a 300× sequencing coverage of each transduced sample. Pooled PCR reactions are analyzed on a 2% TBE gel, bands of expected amplicon sizes are gel purified using the QIAEX II Gel extraction kit (Qiagen, Venlo, Netherlands). The concentrations of purified amplicons are evaluated using the dsDNA BR Assay Kit and QUBIT™ System (Life Technologies, South San Francisco, Calif.) and library quality determined using the Agilent DNA1000Chip and Agilent Bioanalyzer 2100 system (Agilent, Santa Clara, Calif.). Pooled library are sequenced on a MiSeq 2500 (Illumina, San Diego, Calif.).

D. Processing and Analysis of Sequencing Data

The raw sequencing reads are processed by an informatics pipeline such that only reads that align to the target DNA sequence in the FUT8 gene, chr14:65,411,238-65,411,260, are counted. Reads that align to other genomic loci are excluded as they are the result of undesired genomic amplification. The reads that align to this region are analyzed to determine how they differ from the “wild-type” genomic reference sequence. Some fraction of reads has a sequence identical to the reference sequence. Some fraction of reads will have insertions and deletions at the Cas9 cut site that is the result of NHEJ DNA repair by the host cell. Some fraction of reads will contain the sequence signatures of the donor oligonucleotide sequence, these reads are classified as HDR reads. The fraction of sequenced reads that are “wild-type”, “NHEJ” and “HDR” are determined. The relative proportion of these fractions is used to determine whether the fraction of HDR reads is greater than various control samples. Control samples include but are not limited to: HeLa cells that are not transfected with the Cas9-containing plasmid (in this control, there are no expected DSBs in the FUT8 target region so all reads should be “wild-type”), HeLa cells that are transfected with the Cas9-containing plasmid but no donor polynucleotide (in this control, there are DSBs expected in the FUT8 target region, but no donor template for HDR-mediate repair, so all reads should be “wild-type” or “NHEJ”), HeLa cells that are transfected with the donor polynucleotide and a Cas9-containing plasmid where the Cas9 is expressed constitutively rather than with a conditionally active promoter (in this control, there are expected “HDR” reads, but the relative proportion will be lower because Cas9-mediated DSBs occur during stages of the cell cycle where HDR is not favored).

### E. Further Modifications

Other chromosomal loci within HeLa (or other) cells can be modified by this technique. The genomic target DNA sequence and also the sequence to be incorporated at this locus are readily modifiable by one of ordinary skill in the art in view of the teachings of the present specification. This procedure provides data to support use of the Cas9-expressing plasmid systems described herein.

### Example 3

### Identifying DNA Repair Pathway Components

This example describes a screen to determine DNA repair pathway components that, when repressed transiently, facilitate higher levels of HDR relative to the components at the levels they are normally expressed. dCas9 is used as a tool to repress the expression of genes that would inhibit or compete with HDR pathways. As most of these genes are essential, a permanent inhibition would lead to cell death or arrest and an inability to recover HDR outcomes. Repression is relieved by the subsequent transcription of sgRNAs that target dCas9 (see, e.g., FIG. 3 and FIG. 5).

In a candidate-based approach, all genes known to be involved in “error-prone” repair of double-strand DNA breaks (e.g., components of NHEJ and MMEJ pathways) are included. A library of sgRNAsₚᵣₒₘₒₜₑᵣ (comprising, for example, 5 sgRNAs) is designed to target the promoter region of each candidate gene. Each sgRNAₚᵣₒₘₒₜₑᵣ is cloned individually into a vector that contains dCas9 expressed under a constitutive promoter. On the same vector under a separate cell-cycle specific promoter, sgRNAs_{dCas9} designed to extinguish the expression of dCas9 are included.

Orthologous components to generate DSBs can be included on a separate vector or by introduction of dCas9 protein/guide RNA ribonucleoprotein complexes directly into cells. Donor polynucleotides can be introduced in any form.

For example, a plasmid comprising an sgRNAₚᵣₒₘₒₜₑᵣ, designed to target the promoter region of a candidate gene, and cognate dCas9 protein coding sequences under control of a constitutive promoter is introduced into a proliferating cell type (e.g., HEK293 or BJ-hTERT). On the same plasmid under a separate cell-cycle specific promoter (e.g., lncRNA upst:CCNL1:-2767, Hung, T., et al., Nat. Genet. 43:621-629 (2011), see FIG. 4 A-B thereof), sgRNAs_{dCas9} designed to extinguish the expression of dCas9 are included. The plasmid is electroporated into cells with a donor polynucleotide and a plasmid encoding an orthologous Cas9 protein and an sgRNA_{target} to make a DSB at a genomic target DNA sequence.

Each well in this screen contains a separate plasmid containing an sgRNAₚᵣₒₘₒₜₑᵣ targeting dCas9 to a gene involved in error-prone DSB repair. dCas9 and its cognate sgRNAₚᵣₒₘₒₜₑᵣ are expressed constitutively to suppress expression of the gene involved in error-prone DSB repair. The plasmid encoding an orthologous Cas9 protein and sgRNA_{target} to make a DSB is electroporated into the cells in each well. Entry into G₂ phase of the cell cycle leads to the expression of the sgRNAs_{target} from a separate promoter. Extinguishing the expression of dCas9 terminates repression of the candidate gene.

HDR rates are determined by phenotyping (e.g., correction of a cell surface marker or expression of green fluorescence protein by repair of the DSB using sequences from the donor polynucleotide) and by next-generation sequencing (NGS) analysis. Elevated HDR rates relative to controls (e.g., HDR levels in a setting without the repression of end-joining components) provide identification of genes that, when repressed transiently, facilitate HDR.

### Example 4

### Generating a Cell Line with an Integrated Cassette

In this example, a stable cell line containing an expression cassette integrated at a genomic location is generated. A selected gene that, when expression is transiently repressed, facilitates HDR (e.g., a gene discovered in the screen described in Example 3) is used to integrate a large cassette at high efficiency in a predefined locus.

A chimeric antigen receptor (CAR) protein expression cassette is introduced into donor-derived primary T cells. A donor template containing the expression cassette encoding the CAR protein is electroporated into cells with dCas9/sgRNAₚᵣₒₘₒₜₑᵣ ribonucleoprotein complexes that transiently suppresses the selected gene (e.g., a gene discovered in Example 3). An orthogonal Cas9 mRNA and cognate sgRNA_{target} to generate a DSB at a predefined locus are co-electroporated. Delivery of the Cas9 mRNA (versus delivery of the Cas9 protein/sgRNA complex) provides a window for repression to occur before a DSB is generated.

Engineered Class 2 CRISPR-Cas systems, as described herein, for example, such as those described in FIG. 2 and FIG. 3 can be used to alleviate the repression of the selected gene by providing an active Cas protein and cognate sgRNA, wherein the sgRNA targets the Cas protein to cleave the dCas9 coding sequence.

T cells containing the expression cassette are isolated and clonally expanded ex vivo.

### Example 5

### Combined Use of a Cell Cycle Regulated Promoter and Cas9 Protein Depletion Using a Chemically Controlled Degron Tag

In this example, methods described by Natsume, T., et al., Cell Reports 15:210-218 (2016), are modified using the cell cycle specific Class 2 Type II CRISPR-Cas9 systems as described herein to degrade Cas9 protein in a cell cycle specific manner to increase HDR efficiency relative to controls wherein Cas9 protein expression is not coordinated to a selected cell cycle phase (e.g., constitutive expression of Cas9 protein).

A cell line is made that expresses OsTIR1 (an auxin responsive F-box protein derived from Oryza sativa, which forms an efficient ubiquitin ligase with endogenous eukaryotic components) protein in a cell-cycle specific manner (examples of suitable regulatory elements are given in Example 1). The promoter of OsTIR1 is engineered to express the protein during the G₁ phase of the cell cycle, when cells have not replicated their DNA and NHEJ is favored. Cas9 fused to an auxin-inducible degron (AID) is constitutively expressed from a plasmid introduced into the stable cell line expressing OsTIR1 in a cell cycle specific manner. Auxin is present throughout the experiment.

In the presence of auxin, OsTIR1 protein binds to Cas9-AID fusion protein and rapidly degrades the protein. In the absence of expression of the OsTIR1 protein, Cas9-AID forms a ribonucleoprotein complex with a cognate guide RNA that targets the complex to cleave a target nucleic acid sequence. The target is any site where one would like to incorporate information through HDR mechanisms using a donor polynucleotide. For example, a knockout mutation can be created by inserting a stop codon, a mutation within a target nucleic acid sequence can be corrected, a point mutation within a target nucleic acid sequence can be introduced, or a protein can be tagged with a detectable marker (e.g., green fluorescent protein). One advantage of this approach, in addition to the aspect that Cas9 will be present only during cell cycle stages where HDR is favored, is the rate at which Cas9 can be degraded (e.g., compared with Example 1 and Example 2). The protein is the substrate for degradation rather than transcriptional repression which has a longer time frame.

In another configuration, a screen can be performed with candidate genes identified using the method described in Example 3, for example, genes encoding proteins involved in end-joining pathways other than HDR. A cell line is made that expresses OsTIR1 protein in a cell-cycle specific manner. The promoter of OsTIR1 is engineered to express the protein during S and G₂ phases of the cell cycle, when HDR pathways are favored. Proteins involved in end-joining pathways (POI, proteins of interest) that might compete with HDR are endogenously tagged with AID. Endogenous tagging of proteins is achieved by creating a DSB in conditions favorable to HDR and providing a donor polynucleotide containing the AID tag. Auxin is present throughout the experiment.

In the presence of auxin, OsTIR1 binds to POI-AID and rapidly degrades the POI. In the absence of OsTIR1, POI-AID can perform endogenous functions. Cas9 and a cognate guide RNA are introduced into the cell with a donor polynucleotide by methods previously described. A target nucleic acid sequence is any site selected to incorporate information through HDR mechanisms using a donor polynucleotide. The advantage of this approach is the rate at which the POI can be degraded. The protein is the substrate for degradation rather than transcriptional repression which has a longer time frame.

As is apparent to one of skill in the art, various modification and variations of the above embodiments can be made without departing from the spirit and scope of this invention. Such modifications and variations are within the scope of this invention.

What is claimed is:

1. A Class 2 CRISPR-Cas polynucleotide composition comprising:
a first polynucleotide encoding a Cas protein, wherein the first polynucleotide is operably linked to a first regulatory element that is active in response to a first cell state of a eukaryotic host cell.

2. The Class 2 CRISPR-Cas polynucleotide composition of claim 1, further comprising a locus-specific guide polynucleotide encoding a locus-specific guide RNA capable of forming a complex with the Cas protein.

3. The Class 2 CRISPR-Cas polynucleotide composition of claim 2, wherein the locus-specific guide polynucleotide is operably linked to a regulatory element that is active in response to the first cell state of the eukaryotic host cell.

4. The Class 2 CRISPR-Cas polynucleotide composition of claim 3, wherein the first regulatory element is operably linked to a single polynucleotide comprising the first polynucleotide and the locus-specific guide polynucleotide, and wherein a transcript separator sequence is located between the first polynucleotide and the locus-specific guide polynucleotide.

5. The Class 2 CRISPR-Cas polynucleotide composition of claim 1, wherein the first cell state is a transient cell state of the eukaryotic host cell.

6. The Class 2 CRISPR-Cas polynucleotide composition of claim 3, further comprising:
a Cas protein-specific guide polynucleotide encoding a Cas protein-specific guide RNA that is capable of targeting the Cas protein to the first polynucleotide, wherein the Cas protein-specific guide polynucleotide is operably linked to a second regulatory element that is active in response to a second cell state of the eukaryotic host cell.

7. The Class 2 CRISPR-Cas polynucleotide composition of claim 6, wherein the first cell state and the second cell state are different.

8. The Class 2 CRISPR-Cas polynucleotide composition of claim 6, wherein the Cas protein-specific guide polynucleotide encodes multiple copies of the Cas protein-specific guide RNA, wherein sequences encoding the copies of the Cas protein-specific guide RNA are separated by a transcript separator sequence.

9. The Class 2 CRISPR-Cas polynucleotide composition of claim 3, further comprising:
a repressor polynucleotide encoding a repressor protein that is capable of repressing transcription mediated by the first regulatory element, wherein the repressor polynucleotide is operably linked to a non-homologous end-joining (NHEJ) pathway-specific regulatory element that is capable of mediating expression of a protein that drives the NHEJ pathway.

10. The Class 2 CRISPR-Cas polynucleotide composition of claim 9, wherein the first regulatory element further comprises a lacO operator sequence, and the repressor polynucleotide comprises a lac repressor protein coding sequence.

11. The Class 2 CRISPR-Cas polynucleotide composition of claim 1, further comprising:
a first locus-specific guide polynucleotide encoding a locus-specific guide RNA capable of forming a complex with the Cas protein;
a second polynucleotide encoding an inactive Cas (dCas) protein operably linked to a second regulatory element,
a second locus-specific guide polynucleotide encoding a locus-specific guide RNA capable of forming a complex with the dCas protein; and
wherein the first regulatory element and the second regulatory element are both active in response to the first cell state of the eukaryotic host cell.

12. The Class 2 CRISPR-Cas polynucleotide composition of claim 11, wherein the second locus-specific guide RNA comprises a NHEJ pathway-specific guide RNA that is capable of targeting a gene that encodes a protein that drives the NHEJ pathway.

13. The Class 2 CRISPR-Cas polynucleotide composition of claim 1, wherein the first cell state comprises a cell cycle phase.

14. The Class 2 CRISPR-Cas polynucleotide composition of 13, wherein the cell cycle phase is S or G₂.

15. The Class 2 CRISPR-Cas polynucleotide composition of claim 13, wherein the cell cycle phase is G₁, G₀, or M.

16. The Class 2 CRISPR-Cas polynucleotide composition of claim 1, wherein the Cas protein is a Cas9 protein or a Cpf1 protein.

17. The Class 2 CRISPR-Cas polynucleotide composition of claim 3, further comprising:
a donor polynucleotide.

18. One or more vectors comprising the Class 2 CRISPR-Cas polynucleotide composition of claim 3.

19. The one or more vectors of claim 18, wherein the one or more vectors are mammalian expression vectors.

20. The one or more vectors of claim 19, wherein the mammalian expression vector is a lentiviral vector.
